(19) United States
(12) Patent Application Publication
(10) Pub. No.: US 2022/0202967 A1
(43) Pub. Date: Jun. 30, 2022

(54) COMPOSITIONS AND METHODS OF IMMUNODEPLETION FOR THE TREATMENT OF MALIGNANT AND NON-MALIGNANT HEMATOLOGICAL DISEASES

(71) Applicant: ACTINIUM PHARMACEUTICALS, INC., New York, NY (US)

(72) Inventors: Dale Ludwig, Rockaway, NJ (US); Eileen Geoghegan, Mamaroneck, NY (US)

(73) Assignee: ACTINIUM PHARMACEUTICALS, INC., New York, NY (US)

(21) Appl. No.: 17/606,226

(22) PCT Filed: Apr. 24, 2020
(86) PCT No.: PCT/US2020/029887

§ 371 Date: Oct. 25, 2021

Related U.S. Application Data
(60) Provisional application No. 62/838,646, filed on Apr. 25, 2019.

(51) Int. Cl. A61K 51/10; A61P 37/06; A61P 35/00

(52) U.S. Cl. CPC A61K 51/1042; A61K 51/1021; A61P 35/00; A61K 51/1033; A61P 37/06; A61K 51/1027

(57) ABSTRACT

Compositions and methods for transient immunodepletion of specific subsets of a subject's immune cells are disclosed. The methods generally include administering to the subject an effective amount of a radiolabeled antibody against CD19, CD20, CD33, CD38, CD45RA, CD52, or a combination thereof. The effective amount of the radiolabeled antibody depletes at least 50% of the targeted immune cells, and less than 20% of the subject's stem cells. When used alone, these methods may target lymphomas, leukemias, and myelomas, and/or may additionally allow repopulation of non-autoreactive immune cells in patients with an autoimmune disease. When these methods precede certain cell-based therapies, such as adoptive cell therapy and/or hematopoietic stem cell therapy, the methods are able to enhance the outcome of the cell-based therapies while minimizing adverse effects.

### CROSS-REFERENCE TO RELATED APPLICATION

This application claims the benefit of priority to U.S. Provisional Patent Application Ser. No. 62/838,646 filed on Apr. 25, 2019, which is incorporated herein in its entirety.

### FIELD OF THE INVENTION

The present invention relates to compositions that selectively target and deplete cells of specific lineages without targeting stem cells, and methods for transient immunodepletion using these compositions, such as prior to adoptive cell therapy.

### BACKGROUND OF THE INVENTION

Immunodepletion is the process of transiently reducing cells of the immune system while leaving the stem cell compartment intact. Immunodepletion can be a useful and necessary process in the context of adoptive cell therapy, where endogenous host cells must first be depleted prior to the transfer of autologous or allogeneic cells. This phenomenon has been well established in the field of bone marrow transplant, and is also a widespread requirement for the efficacy of other adoptive cell therapies such as CAR-T. These adoptively transferred cells can either be un-manipulated (bone marrow transplant for treatment of blood cancer) or engineered in a way to improve a pathogenic state in the person receiving the adoptively transferred cells (correcting an autoimmune disease by eliminating autoreactive cells before autologous stem cell transplant or engineered CAR-T cells for cancer treatment). Because stem cells are continually generating new hematopoietic progenitor cells of both the myeloid and lymphoid compartment of the immune system, removal of the more differentiated cells is temporary, and these depleted cells are eventually replaced by nascently maturing cells derived from the stem cells. Immunodepletion could also potentially be used without being followed by an adoptive cell therapy, for example, in the case of autoimmune diseases where the differentiated autoreactive cells are depleted and then the functional, non-autoreactive immune cells are repopulated from the healthy stem cells.

Chemotherapy-Based Immunodepletion

It is common to use a combination of highly cytotoxic chemotherapy agents, especially cyclophosphamide and fludarabine, to immunodeplete (i.e., lymphodeplete) patients prior to adoptive cell therapies methods such as CAR-T. These agents reduce lymphoid cell number. However, they are highly toxic. They not only deplete the immune system in a non-targeted manner but may also damage other normal cells and tissues. Not all patients can tolerate them. Further, particularly in CAR T-cell therapy, durable response rates are typically less than 50%. Many patients eventually relapse after receiving CAR T-cell therapy and require further therapeutic intervention or a stem cell transplant (e.g., a bone marrow transplant).

Antibody-Based Immunodepletion

Antibodies have greater cell-targeting specificity than chemotherapeutics. Antibodies to immune cell-specific antigens are therefore of interest as potential substitutes for chemotherapeutics as immunodepleting agents. While naked antibodies may be able to achieve some level of cell killing, a more effective modality to deplete immune cells would be to harness the potent cytotoxic activity of radioactivity in the form of conjugating radionuclides to an antibody or antibody fragment. This may effectively and specifically target the cell killing ability of the radionuclide to cells expressing the protein of interest on the cell surface.

Accordingly, what is needed are alternative methods and therapy protocols which may improve the efficacy and reduce the toxicity of the known immunodepletion regimes, and of adoptive cell therapies such as CAR-T, and/or may reduce certain of the side effects of the current adoptive cell therapy protocols.

### SUMMARY OF THE INVENTION

The present invention provides solutions to the aforementioned problems by providing compositions useful for targeted and transient immunodepletion, and methods of use of these compositions in the treatment of malignant and non-malignant hematological diseases, or prior to administration of an adoptive cell therapy.

The compositions comprise antibodies against antigens (e.g., cell surface markers) that are unique to certain hematological lineages of cells but not others. This differential expression of surface markers can be exploited to selectively deplete cells of specific lineages while leaving other immune cells unaffected, such as stem cells. Exemplary surface markers that may be used to deplete discrete subsets of immune cells without targeting stem cells include any of CD2, CD3, CD5, CD11a, CD11b, CD11c, CD14, CD18, CD19, CD20, CD28, CD33, CD38, CD45RA, CD49a, CD52, CD96, CD116, CD122, CD132, CD152, CD154, CD244, CD272, CD305, CLA, IL-21, B7-HA, or combinations thereof.

Thus, the present invention generally relates to compositions comprising antibodies against any of CD2, CD3, CD5, CD11a, CD11b, CD11c, CD14, CD18, CD19, CD20, CD28, CD33, CD38, CD45RA, CD49a, CD52, CD96, CD116, CD122, CD132, CD152, CD154, CD244, CD272, CD305, CLA, IL-21, B7-HA, or a combination thereof. The antibodies may comprise a radiolabel, such as any of ¹³¹I, ¹²⁵I, ¹²³I, ⁹⁰Y, ¹⁷⁷Lu, ¹⁸⁶Re, ¹⁸⁸Re, ⁸⁹Sr, ¹⁵³Sm, ³²P, ²²⁵Ac, ²¹³Bi, ²¹³Po, ²¹¹At, ²¹²Bi, ²¹³Bi, ²²³Ra, ²²⁷Th, ¹⁴⁹Tb, ¹³⁷Cs, ²¹²Pb, or ¹⁰³Pd.

The present invention further relates to methods for transient immunodepletion of a subset of hematological cells comprising administering to a patient an effective amount of any of the aforementioned compositions. The immunodepletion may be provided before, after, or both before and after, administration of an adoptive cell therapy, such as administration of a population of cells expressing a CAR/TCR or the TIL. The population of cells expressing the CAR/TCR or the TIL may be autologous cells, allogeneic cells derived from another human patient, or xenogeneic cells derived from an animal of a different species.

According to certain aspects of the present invention, the population of cells expressing the CAR/TCR or the TIL may be isolated by leukapheresis, transduced and selected approximately 4 weeks immediately prior to administration, as in the case of autologous cells, or may be isolated from a healthy donor and prepared in advance then stored, such as a frozen preparation, for one or more patients as in the case of so called “off-the-shelf” allogeneic CAR-T cell therapies. The population of cells expressing the CAR/TCR may comprise a population of activated T-cells or natural killer (NK) cells or dendritic cells expressing the CAR/TCR which recognize an antigen. Dendritic cells are capable of antigen presentation, as well as direct killing of tumors. Further, the population of cells may be a pluripotent stem cell population that can be differentiated into a variety of different blood cell types.

According to certain aspects, the radiolabel may be ¹³¹I or ²²⁵AC, wherein an effective amount of an ¹³¹I-labeled antibody may be from 25 mCi to 200 mCi (e.g., 50 mCi, 100 mCi or 150 mCi), and an effective amount of an ²²⁵Ac-labeled antibody may be from 0.1 μCi/kg of subject weight to 5.0 μCi/kg of subject weight.

These methods may improve treatment outcomes for malignant and non-malignant hematological diseases, and/or may lessen side effects associated with adoptive cell therapies, such as neurotoxicity, cytokine release syndrome (CRS), hypogammaglobulinemia, cytopenias, capillary leak syndrome (CLS), macrophage activation syndrome (MAS), tumor lysis syndrome (TLS), and combinations thereof.

The various aspects of the present invention will be realized and attained by means of the combinations specifically outlined in the appended claims. The foregoing general description and the following detailed description and examples of this invention are provided to illustrate various aspects of the present invention, and by no means are to be viewed as limiting any of the described embodiments.

### BRIEF DESCRIPTION OF THE FIGURES

FIG. 1A depicts a prior art adoptive cell therapy protocol which includes leukapheresis, cytoreduction using standard chemotherapeutic agents, CAR/TCR T-cell infusion, and possible myeloconditioning in preparation for optional hematopoietic stem cell transplantation (HSCT).

FIG. 1B depicts an autologous or allogeneic adoptive cell therapy treatment of the present invention which includes administration of a radiolabeled antibody instead of, or in addition to, the standard cytoreduction agents.

FIG. 1C depicts an autologous or allogeneic adoptive cell therapy treatment of the present invention which includes administration of a radiolabeled antibody as a myeloconditioning agent prior to HSCT.

FIG. 1D depicts an autologous or allogeneic adoptive cell therapy treatment of the present invention which includes administration of a radiolabeled antibody instead of, or in addition to, the standard cytoreduction agents, and administration of a radiolabeled antibody as a myeloconditioning agent prior to HSCT.

FIG. 1E depicts pharmacokinetic data demonstrating exemplary clearance and dosing times for a lymphodepletion protocol according to the presently disclosed invention.

FIG. 2 shows the median change in absolute neutrophil count following dosing with ¹³¹I-anti-CD45 (i.e., ¹³¹I-BC8).

FIGS. 3A-3H show results of immune cell analysis following ¹³¹I-anti-CD45 antibody targeted immunodepletion in a mouse model using surrogate anti-CD45 antibody 30F11.

FIGS. 4A-4E show results from immunophenotyping of immune cell populations following ¹³¹I-anti-CD45 antibody targeted immunodepletion in mice.

FIG. 5A shows depletion of splenic T-reg cells, FIG. 5B shows depletion of myeloid derived suppressor cells (MDSC), and FIG. 5C shows preservation of bone marrow HSC after targeted immunodepletion with ¹³¹I-anti-CD45 antibody in mice.

FIG. 6 shows selected published trials of autologous anti-CD19 CAR T-cell therapy for patients with B-cell non-Hodgkin's lymphoma (NHL).

FIG. 7 shows a schematic of preclinical studies of the effects in mice of low dose ¹³¹I-anti-CD45 radioimmunotherapy (surrogate 30F11) investigating the immunodepletive response on particular immune cell types. Controls include chemotherapeutic lymphodepletive treatments, cyclophosphamide (Cy) or cyclophosphamide/fludarabine (Flu/Cy), and no lymphodepletive treatment.

FIG. 8 shows a preclinical model of adoptive T-cell transfer following anti-CD45 radioimmunotherapy-mediated conditioning/immunodepletion in mice. In this model, E.G7 lymphoma tumor-bearing mice will be conditioned by a single selected dose of ¹³¹I-anti-CD45 radioimmunotherapy prior to adoptive cell transfer of OVA-specific CD8+ T-cells and monitored for engraftment of the transferred cells and resulting anti-tumor response.

FIG. 9 shows clinical data from a low dose ¹³¹I-BC8 study demonstrating lymphodepletion in human patients.

FIG. 10 shows pharmacokinetic data demonstrating the clearance rate (<25 cGy) of a 100 mCi infusion of ¹³¹I-BC8 in human patients.

FIG. 11 shows pharmacokinetic data demonstrating the cumulative dose of ¹³¹I-BC8 to spleens of human patients after administration of 100 mCi infusion of the ¹³¹I-BC8.

FIG. 12 shows blood clearance of ¹³¹I-BC8 from human patients after administration of 100 mCi infusion of the ¹³¹I-BC8.

FIGS. 13A and 13B show results from ¹³¹I-anti-CD45 targeted immunodepletion prior to adoptive cell therapy with OT I OVA reactive T cells on day 4 for E.G7-OVA tumor engrafted mice.

### DETAILED DESCRIPTION OF THE INVENTION

This invention provides radiolabeled antibody-based methods for immunodepleting a subject, and related compositions and articles of manufacture. When these methods precede certain cell-based therapies, the methods are able to enhance the outcome of the cell-based therapies while minimizing adverse effects.

### Definitions

In this application, certain terms are used which shall have the meanings set forth as follows.

The singular forms “a,” “an,” “the” and the like include plural referents unless the context clearly dictates otherwise. Thus, for example, reference to “an” antibody includes both a single antibody and a plurality of different antibodies.

The term “about” when used before a numerical designation, e.g., temperature, time, amount, and concentration, including a range, indicates approximations which may vary by ±10%, ±5%, or ±1%.

As used herein, “administer”, with respect to an antibody, means to deliver the antibody to a subject's body via any known method suitable for antibody delivery. Specific modes of administration include, without limitation, intravenous, transdermal, subcutaneous, intraperitoneal, intrathecal and intra-tumoral administration. Exemplary administration methods for antibodies may be as substantially described in International Publication No. WO 2016/187514, incorporated by reference herein.

In addition, in this invention, antibodies can be formulated using one or more routinely used pharmaceutically acceptable carriers. Such carriers are well known to those skilled in the art. For example, injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's).

As used herein, the term “antibody” includes, without limitation, (a) an immunoglobulin molecule comprising two heavy chains and two light chains and which recognizes an antigen; (b) polyclonal and monoclonal immunoglobulin molecules; (c) monovalent and divalent fragments thereof (e.g., Fab, di-Fab), and (d) bi-specific forms thereof. Immunoglobulin molecules may derive from any of the commonly known classes, including but not limited to IgA, secretory IgA, IgG and IgM. IgG subclasses are also well known to those in the art and include, but are not limited to, human IgG1, IgG2, IgG3 and IgG4. Antibodies can be both naturally occurring and non-naturally occurring (e.g., IgG-Fc-silent). Furthermore, antibodies include chimeric antibodies, wholly synthetic antibodies, single chain antibodies (e.g., scFv), single and double domain antibodies (e.g., VHH), and fragments thereof. Antibodies may be human, humanized or nonhuman.

“Monoclonal antibody” refers to a preparation of antibody molecules of single molecular composition. A monoclonal antibody composition displays a single binding specificity and affinity for a particular epitope, or in a case of a multi-specific monoclonal antibody, a binding specificity to two or more distinct epitopes. “Monoclonal antibody” therefore refers to an antibody population with single amino acid composition in each heavy and each light chain, except for possible well-known alterations such as removal of C-terminal lysine from the antibody heavy chain. Monoclonal antibodies may have heterogeneous glycosylation within the antibody population. Monoclonal antibodies may be monospecific or multi-specific, or monovalent, bivalent or multivalent.

As used herein, an “anti-CDXX antibody” is an antibody that specifically binds to any available epitope of CDXX, wherein the XX may be 2, 3, 5, 11a, 11b, 11c, 14, 18, 19, 20, 28, 33, 38, 45RA, 49a, 52, 96, 116, 122, 132, 152, 154, 244, 272, or 305 (i.e., CD2, CD3, CD5, CD11a, CD11b, CD11c, CD14, CD18, CD19, CD20, CD28, CD33, CD38, CD45RA, CD49a, CD52, CD96, CD116, CD122, CD132, CD152, CD154, CD244, CD272, or CD305). CDXX may also refer to CLA, IL-21, B7-HA, CLA, IL-21, or B7-HA. According to certain preferred aspects, the XX may be 19, 20, 33, 38, 45, or 52 (i.e., CD19, CD20, CD33, CD38, CD45RA, and CD52).

An anti-CDXX antibody may be a bispecific antibody that binds to two different epitopes, wherein the epitopes may be at least any one of the CD19, CD20, CD33, CD38, CD45RA, and CD52, and another relevant epitope; or may be two different epitopes of a single cell surface target (two different epitopes of any one of CD19, CD20, CD33, CD38, CD45RA, or CD52). The bispecific antibody may be an antibody that targets a lymphoid derived cell, but not a stem cell; and/or a myeloid derived cell but not a stem cell. The lymphoid target may be CD3, CD2, CD28, CD96, CD122, CD152, or CD154; and/or the myeloid target may be CD11b, CD11c, CD14, CD33, or CD116. The bispecific antibody may be a recombinant antibody, a monoclonal antibody, a chimeric antibody, a humanized antibody, a human antibody, or an antibody fragment.

The term “specific binding” refers the property of having a high binding affinity of at least 10⁶ M⁻¹, and usually between about 10⁶ M⁻¹ and about 10⁸M⁻¹.

As used herein, “cancer” includes, without limitation, a solid cancer (e.g., a tumor) and a hematologic malignancy.

As used herein, “depleting” or “targeted depletion”, with respect to a subject's immune cells shall mean to lower the population of at least one type of the subject's immune cells (i.e., targeted immune cells) by administration of a specific antibody according to aspects of the present invention. For example, the targeted immune cells may include at least B-cells such as pro-B, pre-B, and plasma cells (e.g., cells comprising CD19, CD20 antigens); myeloid-derived cells such as common myeloid progenitor, myeloid dendritic cells, mast cells, granulocyte macrophage progenitors, monocytes, macrophages, myeloblasts, neutrophils, eosinophils, and basophils (e.g., CD33); natural killer cells, B cells, plasma cells, T cells, T regs, dendritic cells, common myeloid progenitors, granulocyte macrophage progenitors, myeloblasts, basophils, monocytes, megakaryocyte erythroid progenitors, erythrocytes, megakaryocytes (e.g., CD38); lymphoid progenitors, double negative (CD4− CD8−) T cells, B cells, NK cells, granulocyte/monocyte precursor, neutrophils, activated monocyte/macrophage (e.g., CD45RA); mature B and T lymphocytes, monocytes, NK cells, and dendritic cells (e.g., CD52). According to preferred aspects, depleting the subject's immune cells means to lower the population of immune cells without also lowering the population of stem cells, such as hematopoietic stem cells (“HSCs”, i.e., multi-potential hematopoietic stem cells, also referred to as hemocytoblasts).

According to certain preferred aspects of the presently disclosed invention, a subject's immune cell decrease or depletion is determined by measuring a specific population of immune cells in the subject's peripheral blood. For example, the subject's peripheral blood lymphocyte population is depleted if the population of at least one type of the subject's peripheral blood lymphocytes is lowered by no more than 99%. For example, a subject's lymphocytes are depleted if the subject's peripheral blood T-cell level is lowered by 50%, the subject's peripheral blood NK cell level is lowered by 40%, and/or the subject's peripheral blood B cell level is lowered by 30%. In this example, the subject's lymphocytes are depleted even if the level of another immune cell type, such as neutrophils, is not lowered. According to certain aspects, depleting a subject's lymphocytes is reflected by a peripheral blood lymphocyte population reduction of at least 30%, 40%, 50%, 60%, 70%, 80%, 90%, 95%, or 99%. According to certain aspects, the subject's HSC population is depleted by no more than 20%, such as no more than 10%.

Methods for measuring peripheral blood immune cell populations are routine. They include, for example, flow cytometry on whole blood samples to determine counts of specific cell types based on labeling with a fluorescent antibody directed against a specific a cell surface marker. For example, lymphocyte counts may be determined using markers such as CD45, CD4 or CD8, and neutrophil counts may be determined using a marker such as Ly6G. Methods for measuring HSC populations are routine, and include, for example, the use of flow cytometry and fluorescent antibodies directed against a cell surface markers such as Lin, CD34, CD38, CD43, CD45RO, CD45RA, CD59, CD90, CD109, CD117, CD133, CD166, and HLA DR (i.e., markers that recognize stem cells, e.g., CD34, and those that specifically exclude stem cells, e.g., CD45RA).

As used herein, an amount of a radiolabeled anti-CDXX antibody, when administered, is “effective” if the subject's targeted immune cells are depleted, such as by at least 30%, 40%, 50%, 60%, 70%, 80%, 90%, 95%, or 99%. An amount of radiolabeled anti-CDXX antibody, when administered, is “effective” if the subject's targeted immune cells are depleted without depletion of the subject's stem cells, or with less than 20% or 10% reduction in the subject's stem cells.

According to certain aspects, when the radiolabeled anti-CDXX antibody is ¹³¹I-labeled, the effective amount is below, for example, 300 mCi (i.e., where the amount of ¹³¹I-anti-CDXX administered to the subject delivers a total body radiation dose of below 300 mCi). According to certain aspects, when the antibody is ¹³¹I-anti-CDXX, the effective amount is below 250 mCi, below 200 mCi, below 150 mCi, below 100 mCi, below 50 mCi, below 40 mCi, below 30 mCi, below 20 mCi or below 10 mCi. According to certain aspects, when the antibody is ¹³¹I-anti-CDXX, the effective amount is from 1 mCi to 10 mCi, from 1 mCi to 200 mCi, from 10 mCi to 20 mCi, from 10 mCi to 30 mCi, from 10 mCi to 40 mCi, from 10 mCi to 50 mCi, from 10 mCi to 100 mCi, from 10 mCi to 150 mCi, from 10 mCi to 200 mCi, from 20 mCi to 30 mCi, from 30 mCi to 40 mCi, from 40 mCi to 50 mCi, from 50 mCi to 100 mCi, from 50 mCi to 150 mCi, from 50 mCi to 200 mCi, from 60 mCi to 140 mCi, from 70 mCi to 130 mCi, from 80 mCi to 120 mCi, from 90 mCi to 110 mCi, from 100 mCi to 150 mCi, from 150 mCi to 200 mCi, or from 200 mCi to 250 mCi. According to certain aspects, when the antibody is ¹³¹I-anti-CDXX, the effective amount is from 10 mCi to 120 mCi, from 20 mCi to 110 mCi, from 25 mCi to 100 mCi, from 30 mCi to 100 mCi, from 40 mCi to 100 mCi, or from 75 mCi to 100 mCi. According to certain aspects, when the antibody is ¹³¹I-anti-CDXX, the effective amount is 1 mCi, 10 mCi, 20 mCi, 30 mCi, 40 mCi, 50 mCi, 60 mCi, 70 mCi, 80 mCi, 90 mCi, 100 mCi, 110 mCi, 120 mCi, 130 mCi, 140 mCi, 150 mCi, or 200 mCi.

According to certain aspects, when the radiolabeled anti-CDXX antibody is ²²⁵Ac-labeled, the effective amount is below, for example, 5.0 μCi/kg (i.e., where the amount of ²²⁵Ac-anti-CDXX administered to the subject delivers a radiation dose of below 5.0 μCi per kilogram of subject's body weight). According to certain aspects, when the antibody is ²²⁵Ac-anti-CDXX, the effective amount is below 4.5 μCi/kg, 4.0 μCi/kg, 3.5 μCi/kg, 3.0 μCi/kg, 2.5 μCi/kg, 2.0 μCi/kg, 1.5 μCi/kg, 1.0 μCi/kg, 0.9 μCi/kg, 0.8 μCi/kg, 0.7 μCi/kg, 0.6 μCi/kg, 0.5 μCi/kg, 0.4 μCi/kg, 0.3 μCi/kg, 0.2 μCi/kg, 0.1 μCi/kg or 0.05 μCi/kg. According to certain aspects, when the antibody is ²²⁵Ac-anti-CDXX, the effective amount is from 0.05 μCi/kg to 0.1 μCi/kg, from 0.1 μCi/kg to 0.2 μCi/kg, from 0.2 μCi/kg to 0.3 μCi/kg, from 0.3 μCi/kg to 0.4 μCi/kg, from 0.4 μCi/kg to 0.5 μCi/kg, from 0.5 μCi/kg to 0.6 μCi/kg, from 0.6 μCi/kg to 0.7 μCi/kg, from 0.7 μCi/kg to 0.8 μCi/kg, from 0.8 μCi/kg to 0.9 μCi/kg, from 0.9 μCi/kg to 1.0 μCi/kg, from 1.0 μCi/kg to 1.5 μCi/kg, from 1.5 μCi/kg to 2.0 μCi/kg, from 2.0 μCi/kg to 2.5 μCi/kg, from 2.5 μCi/kg to 3.0 μCi/kg, from 3.0 μCi/kg to 3.5 μCi/kg, from 3.5 μCi/kg to 4.0 μCi/kg, from 4.0 μCi/kg to 4.5 μCi/kg, or from 4.5 μCi/kg to 5.0 μCi/kg. According to certain aspects, when the antibody is ²²⁵Ac-anti-CDXX, the effective amount is 0.05 μCi/kg, 0.1 μCi/kg, 0.2 μCi/kg, 0.3 μCi/kg, 0.4 μCi/kg, 0.5 μCi/kg, 0.6 μCi/kg, 0.7 μCi/kg, 0.8 μCi/kg, 0.9 μCi/kg, 1.0 μCi/kg, 1.5 μCi/kg, 2.0 μCi/kg, 2.5 μCi/kg, 3.0 μCi/kg, 3.5 μCi/kg, 4.0 μCi/kg or 4.5 μCi/kg.

For an antibody labeled with a radioisotope, the majority of the composition administered to a subject typically consists of non-labeled antibody, with the minority being the labeled antibody. The ratio of labeled to non-labeled antibody can be adjusted using known methods. Thus, according to certain aspects of the present invention, the radiolabeled antibody may comprise a labeled fraction and an unlabeled fraction. The radiolabeled antibody may be provided as a single dose, wherein the single dose of the radiolabeled antibody may comprise the antibody in an amount of from 0.1:10 to 1:1 labeled:unlabeled. A total protein amount may be up to 60 mg, such as 5 mg to 45 mg, or a total protein amount may be 0.2 mg/kg patient weight to 0.6 mg/kg patient weight.

The adoptive cell therapy may include administration of cells expressing a chimeric antigen receptor (CAR), or a T-cell receptor (TCR), or may include tumor-infiltrating lymphocytes (TIL). The population of cells expressing the CAR/TCR may comprise a population of activated T-cells or natural killer (NK) cells or dendritic cells expressing the CAR/TCR which recognize an antigen. Dendritic cells are capable of antigen presentation, as well as direct killing of tumors. The population of cells expressing the CAR/TCR may comprise a population of gene-edited cells.

As used herein, the term “gene-edited” CAR T-cell is synonymous with the terms “genetically engineered” CAR T-cell and “engineered” CAR T-cell. A gene-edited CAR T-cell that “fails to properly express” a checkpoint receptor (e.g., PD1, Lag3 or TIM3) does not express the full-length, functional checkpoint receptor. For example, a gene-edited CAR T-cell that fails to properly express PD1 may fail to do so because, without limitation, (i) the cell's PD1 gene has been ablated, or (ii) the cell's PD1 gene has been otherwise altered so as not to yield a fully or even partially functional PD1 product. In other words, according to certain aspects, a gene-edited CAR T-cell that fails to properly express PD1 may fail to do so because the cell's PD1 gene has been altered to diminish PD1 expression. Similarly, a gene-edited CAR T-cell that “fails to properly express” a T-cell receptor does not express the full-length, functional T-cell receptor.

According to certain aspects, the functional endogenous T-cell receptor is replaced through editing by a “knock-in” to the native TCR locus of an exogenously transduced CAR or recombinant TCR. The gene-edited CAR T-cells may include, without limitation, the following: (i) allogeneic gene-edited CAR T-cells that fail to properly express PD1 but do properly express all other checkpoint receptors and T-cell receptors; (ii) allogeneic gene-edited CAR T-cells that fail to properly express a particular T-cell receptor but do properly express all checkpoint receptors and all other T-cell receptors; and (iii) allogeneic gene-edited CAR T-cells that fail to properly express PD1 and fail to properly express a particular T-cell receptor, but do properly express all other checkpoint receptors and all other T-cell receptors.

Examples of T-cell gene editing to generate allogeneic, universal CAR T-cells include the work of Eyquem and colleagues (Eyquem, et. al., 2017, Nature. 543:113-117). In that study, the endogenous T-cell receptor alpha constant locus (TRAC) was effectively replaced by a recombinant CAR gene construct. In the method, the recombinant CAR was placed under the control of the cell's native TCR regulatory signals. By this same strategy, CARs or recombinant TCRs may be effectively inserted by knock-in into the T-cell receptor beta constant gene locus (TRBC) or into the beta-2 microglobulin (B2M) MHC-I-related gene locus, known to be expressed in all T-cells. Another example includes the work of Ren and colleagues (Ren, et. al., 2017, Clin. Cancer Res 23:2255-2266). Recognizing that checkpoint receptors are immune-suppressive and may blunt the stimulation of exogenous autologous or allogeneic CAR T-cells, this group exploited CRISPR/cas9 technology to ablate the endogenous TCR α and β loci (TRAC and TRBC) and the B2M gene, while also silencing the endogenous PD1 gene. With this approach, the engineered cells did not elicit graft-versus-host disease but did resist immune checkpoint receptor suppression.

A “hematologic malignancy” or “malignant hematological disease”, also known as a blood cancer, is a cancer that originates in blood-forming tissue, such as the bone marrow or other cells of the immune system. Hematologic malignancies include, without limitation, leukemias (such as acute myeloid leukemia (AML), acute promyelocytic leukemia, acute lymphoblastic leukemia (ALL), acute mixed lineage leukemia, chronic myeloid leukemia, chronic lymphocytic leukemia (CLL), hairy cell leukemia and large granular lymphocytic leukemia), myelodysplastic syndrome (MDS), myeloproliferative disorders (polycythemia vera, essential thrombocytosis, primary myelofibrosis and chronic myeloid leukemia), lymphomas, multiple myeloma, MGUS and similar disorders, Hodgkin's lymphoma, non-Hodgkin's lymphoma (NHL), primary mediastinal large B-cell lymphoma, diffuse large B-cell lymphoma, follicular lymphoma, transformed follicular lymphoma, splenic marginal zone lymphoma, lymphocytic lymphoma, T-cell lymphoma, and other B-cell malignancies.

“Solid cancers” include, without limitation, bone cancer, pancreatic cancer, skin cancer, cancer of the head or neck, cutaneous or intraocular malignant melanoma, uterine cancer, ovarian cancer, prostate cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, pediatric tumors, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, environmentally-induced cancers including those induced by asbestos.

A “non-malignant hematological disease” or “non-cancerous disorder” includes, without limitation, hemoglobinopathies (e.g., SCD and β-thalassemia), congenital immunodeficiencies (e.g., SCID and Fanconi's anemia) and viral infections (e.g., HIV infection). According to certain aspects, the disorder is SCD and the therapy is genetically edited β-globin hematopoietic stem cell therapy. According to certain aspects, the disorder is SCID and the therapy is genetically edited hematopoietic stem cell therapy, wherein the edited gene is the common gamma chain (γc) gene, the adenosine deaminase (ADA) gene and/or the Janus kinase 3 (JAK3) gene. The stem cell therapies can be allogeneic or autologous, for example.

As used herein, the term “subject” includes, without limitation, a mammal such as a human, a non-human primate, a dog, a cat, a horse, a sheep, a goat, a cow, a rabbit, a pig, a rat and a mouse. Where the subject is human, the subject can be of any age. For example, the subject can be 60 years or older, 65 or older, 70 or older, 75 or older, 80 or older, 85 or older, or 90 or older. Alternatively, the subject can be 50 years or younger, 45 or younger, 40 or younger, 35 or younger, 30 or younger, 25 or younger, or 20 or younger. For a human subject afflicted with cancer, the subject can be newly diagnosed, or relapsed and/or refractory, or in remission.

As used herein, a “suitable time period” after administering a radiolabeled antibody to a subject and before performing adoptive cell therapy on the subject is a time period sufficient to permit the administered antibody to deplete the subject's lymphocytes and/or for the subject's lymphocytes to remain depleted. According to certain aspects, the suitable time period is fewer than 10 days, fewer than 9 days, fewer than 8 days, fewer than 7 days, fewer than 6 days, fewer than 5 days, fewer than 4 days, or fewer than 3 days. According to certain aspects, the suitable time period is 1 day, 2 days, 3 days, 4 days, 5 days, 6 days, 7 days, 8 days, 9 days, 10 days, 11 days, 12 days, 13 days, 14 days, 15 days, or greater than 15 days.

As used herein, a “radioisotope” can be an alpha-emitting isotope, a beta-emitting isotope, and/or a gamma-emitting isotope. Examples of radioisotopes include the following: ¹³¹I, ¹²⁵I, ¹²³I, ¹⁷⁷Lu, ¹⁸⁶Re, ¹⁸⁸Re, ⁸⁹Sr, ¹⁵³Sm, ³²P, ²²⁵Ac, ²¹³Bi, ²¹³Po, ²¹¹At, ²¹²Bi, ²¹³Bi, ²²³Ra, ²²⁷Th, ¹⁴⁹Tb, ¹³⁷Cs, ²¹²Pb, or ¹⁰³Pd. Methods for affixing a radioisotope to an antibody (i.e., “labeling” an antibody with a radioisotope) are well known. Certain of these methods are described, for example, in International Publication No. WO 2017/155937.

As used herein, “treating” a subject afflicted with a malignant or nonmalignant hematological disease shall include, without limitation, (i) slowing, stopping or reversing the disease's progression, (ii) slowing, stopping or reversing the progression of the disease's symptoms, and/or (iii) reducing the likelihood of the disease's recurrence. According to certain aspects, treating a subject afflicted with a cancer means (i) reversing the cancer's progression, ideally to the point of eliminating the cancer, and/or (ii) reversing the progression of the cancer's symptoms, ideally to the point of eliminating the symptoms, and/or (iii) reducing or eliminating the likelihood of relapse (i.e., consolidation, which ideally results in the destruction of any remaining cancer cells).

Throughout this application, various publications are cited. The disclosure of these publications is hereby incorporated by reference into this application to describe more fully the state of the art to which this invention pertains. Unless otherwise defined, all technical and scientific terms used herein have the same meaning as commonly understood by one of ordinary skill in the art to which the present invention belongs. Although methods and materials similar or equivalent to those described herein can be used in the practice or testing described herein, suitable methods and materials are described below.

Aspects of the Invention

Targeted Immunodepletion

The present invention solves an unmet need in the art by providing an unexpectedly superior way to transiently immunodeplete specific targeted subsets of immune cells. Cells of the hematopoietic system often express specific surface markers that appear on certain lineages of cells but not others. This differential expression of surface markers can be exploited to selectively deplete cells of specific lineages while leaving other immune cells unaffected. Some potentially useful surface markers that could effectively deplete discrete subsets of immune cells without targeting stem cells include at least: CD19, CD20, CD33, CD38, CD45RA, and CD52.

CD19 is a membrane receptor that is expressed early in B cell differentiation and continues to be expressed until the B cells are triggered to terminally differentiate (i.e., first expressed on pro-B cells and through development to plasma cells). CD19 is part of a protein complex found on the cell surface of B lymphocytes. The protein complex includes CD19, CD21 (complement receptor, type 2), CD81 (TAPA-1), and CD225 (Leu-13). CD19 is an important regulator of transmembrane signals in B cells. An increase or decrease in the cell surface density of CD19 affects B cell development and function, resulting in diseases such as autoimmunity or hypogammaglobulinemia. An exemplary commercially available antibody against CD19 includes blinatumumab.

CD20 is a transmembrane protein expressed on the surface of B lymphocytes. It is expressed during the development of a B lymphocyte from the early pre-B stage until terminal differentiation into plasmocytes, a stage at which CD20 expression disappears. CD20 is also expressed on malignant B cells. In particular, CD20 is expressed on greater than 90% of B cell non-Hodgkin's lymphoma (NHL) cells and over 95% B-type Chronic Lymphocytic Leukemia (B-CLL) cells. While the function is unknown, it is thought to be involved in B-cell activation, regulation of B-cell growth, and transmembrane calcium flux. An exemplary commercially available antibody against CD20 includes rituximab.

CD33 is a type I transmembrane receptor glycoprotein that may function as a sialic acid-dependent cell adhesion molecule. Expressed on early myeloid progenitors, myeloid dendritic cells, mast cells, granulocyte macrophage progenitors, monocytes, macrophages, myeloblasts, neutrophils, eosinophils, and basophils, CD33 is not expressed on stem cells. Exemplary commercially available antibodies against CD33 include lintuzumab (HuM195), gemtuzumab, or vadastuximab.

CD38 is a type II transmembrane glycoprotein with a long C-terminal extracellular domain and a short N-terminal cytoplasmic domain. The CD38 protein is a bi-functional ectoenzyme that can catalyze the conversion of NAD⁺ into cyclic ADP-ribose (cADPR), and cADPR into ADP-ribose, and thus regulates extracellular NAD⁺ concentrations. Moreover, cADPR has been shown to be a second messenger for intracellular Ca²⁺ mobilization. Thus, CD38 may be an essential component for regulation of intracellular Ca²⁺ flux. CD38 has been described on epithelial/endothelial cells of different origin, including glandular epithelium in prostate, islet cells in pancreas, ductal epithelium in glands, including parotid gland, bronchial epithelial cells, cells in testis and ovary, and tumor epithelium in colorectal adenocarcinoma. Exemplary commercially available antibodies against CD38 include daratumumab, MOR202, or SAR650984.

CD45 is a protein expressed exclusively on cells of the hematopoietic system, including on stem cells. A pan-CD45 antibody conjugated to 131-iodine (¹³¹I) has already been exploited for complete ablation of hematopoietic cells prior to bone marrow transplant in many clinical trials. Additionally, a pan-CD45 antibody has been contemplated as a good candidate for immunodepletion when used at low doses, so that it preferentially depletes more differentiated cells that have higher levels of CD45 expression. There are multiple isoforms of CD45, including CD45RO (pan specific), CD45RA, CD45RB. Ideally, a CD45 targeting strategy would use an isoform of CD45 that is not expressed on stem cells, such as CD45RA. Therefore, a radiolabeled CD45RA antibody would deplete the more differentiated immune cells but spare stem cells.

CD52 is a membrane glycoprotein found on mature lymphocytes, but not on the stem cells from which these lymphocytes are derived. The protein is expressed on more than 95% of peripheral blood lymphocytes and is found at a higher density on T lymphocytes than B lymphocytes. An exemplary commercially available antibody against CD52 includes alemtuzumab (Lemtrada, Campath).

These targets, and the cells they may deplete are listed in Table 1 (i.e., CD19, CD20, CD33, CD38, CD45RA, and CD52). Additional targets that may be useful in various aspects of the present invention, and the cells they may deplete are listed in Table 2 (i.e., CD2, CD5, CD11a, CD18, CD49a, CD122, CD132, CD244, CD272, CD305, CLA, IL-21 receptor, and B7-HA).

Additionally, using a combination of targets may also allow for rational depletion of multiple cell types in a therapeutically useful manner. For example, creation of a bispecific antibody where one arm binds a lymphoid marker and the other binds a myeloid marker, neither of which are present on stem cells, would be an ideal immunodepletion target. Such a potential combination strategy could include a bispecific targeting CD3, and CD11c. Targeting CD3 would eliminate T cells, while targeting CD11c would deplete many myeloid cells including monocytes and myeloid dendritic cells, and since neither of these markers is expressed on stem cells, would result in transient depletion of the more differentiated immune cells and eventual repopulation of the hematopoietic system by stem cells. Other potential markers of a subset or all lymphoid cells include: CD3, CD2, CD28, CD96, CD122, CD152, CD154. Potential markers of a subset or all myeloid cells include: CD11b, CD11c, CD14, CD33, CD116. Any combination of the lymphoid or myeloid markers listed could potentially be combined for rational depletion of desired immune cell subsets.

Treatment with radiolabeled antibodies against such cell surface markers provides an effective means to specifically target the cell killing ability of the radionuclide to cells expressing the protein of interest on the cell surface.

Accordingly, this invention employs a radiolabeled anti-CDXX antibody such as a radiolabeled anti-CD19, or anti-CD20, or anti-CD33, or anti-CD38, or anti-CD45RA, or anti-CD52 antibody, or a combination thereof.

TABLE 1

| | | | Dendritic | NK | Macrophage | | |
|---|---|---|---|---|---|---|---|
| Target | T Cell | B Cell | Cell | Cell | /Monocyte | Granulocyte | Brief Description |
| CD19 | | + | + | | | | pro-B cells and through development to plasma cells, some expression on |
| | | | | | | | denritic cells |
| CD20 | + | + | | | | | pre-B stage until terminal differentiation into plasmocytes, a subset of T |
| | | | | | | | cells express |
| CD33 | | | + | | + | + | Early myeloid progenitors, myeloid dendritic cells, mast cells, granulocyte |
| | | | | | | | macrophange progenitors, monocytes, macrophages, myeloblasts. |
| | | | | | | | neufrophils, eosinophils, and basophils |
| CD38 | + | + | + | + | + | + | Natural killer cells, B cells, plasma cells, T cells, T regs, dendritic cells, |
| | | | | | | | common myeloid proenitors, granulocyte macrophange progenitors, |
| | | | | | | | myeloblasts, basophils, monocytes, megakaryocyte erythroid progenitors, |
| | | | | | | | erythrocytes, megakarcocytes |
| CD45RA | + | + | | + | + | + | Lymphoid progenitors, double negative (CD4-, CD8-) T cells, B cells, NK |
| | | | | | | | cells, granufocyte/monocyte precursor, neutrophils, activated |
| | | | | | | | monocyte/macrophange |
| CD52 | + | + | + | + | + | | Mature B and T lymphocytes, monocytes, NK cells and dendritic cells |

TABLE 2

| | | | Dendritic | NK | Macrophage | | |
|---|---|---|---|---|---|---|---|
| Target | T Cell | B Cell | Cell | Cell | /Monocyte | Granulocyte | Brief Description |
| CD2 | + | + | | + | | − | Good marker of T cells and NK cells, only expressed on subset of B cells. |
| CD5 | + | + | | − | − | − | Increasing expression on T cells as they mature/are selected in thymus. |
| | | | | | | | Expressed on subset of B cells. |
| CD11a | + | + | | + | + | + | pan-leukocyte marker and is expressed by B- and T-lymphocytes, |
| | | | | | | | monocytes, marcophages, neutrophils, basophils, and eosinophils. |
| CD18 | + | + | + | + | + | + | All leukocytes, and platelets. |
| CD49a | + | - | | + | + | - | Broad leukocyte expression. |
| CD122 | + | + | | + | + | + | T and B cells, NK cells, monocytes, macrophages. |
| CD132 | + | + | +* | + | + | + | *BD is blank for dendritic cells but they do appear to express it. |
| CD244 | + | - | + | + | + | + | AKA SLAM. Expressed on antigen-experienced T cells, NK cells, DCs, |
| | | | | | | | and MDSCs. Good target since found to be associated with MDSCs. |
| CD272 | + | + | + | − | + | − | AKA BTLA. Constitutively expressed on most CD4+ and CD8+ T cells and |
| | | | | | | | its expression progressively decreases upon T cell activation. |
| CD305 | + | + | + | + | + | + | AKA LAIR1. Appears to have a good widespread expression on immune |
| | | | | | | | cells. But, also expressed on CD34+ HSC. |
| CLA | + | + | | + | + | + | T cells in skin, subsets of preipheral blood memory T cells, NK cells, |
| | | | | | | | memory B cells and dendritic cells as well as on monocytes, granulocytes, |
| | | | | | | | and activated endothelial cells. Also be detected on some peripheral T and |
| | | | | | | | NK cells, as well as monocytes, neutrophils and CD1a+ dendritic cells. |
| IL-21 | + | + | + | + | + | + | Produced by CD4+ T cells and mediates the proliferation, differentiation and |
| receptor | | | | | | | effector functions of cells expressing IL-21R such as B cells, T cells, NK cells, |
| | | | | | | | dendritic cells and other populations of myeloid cells. |
| B7-H4 | + | + | + | | + | + | Activated T cells, B cells, monocytes, and dendritic cells. |

The invention further provides radiolabeled antibodies such as those listed in Table 2, which include a radiolabeled antibody such as a radiolabeled anti-CD2, or anti-CD5, or anti-CD11a, or anti-CD18, or anti-CD49a, or anti-CD122, or anti-CD132, or anti-CD244, or anti-CD272, or anti-CD305, or anti-CLA, or anti-IL-21 receptor, or anti-B7-HA, or combination thereof.

The invention further yet provides radiolabeled antibodies that specifically target lymphoid or myeloid cells, such as radiolabeled anti-CD2, or anti-CD3, or anti-CD11b, or anti-CD11c, or anti-CD14, or anti-CD28, or anti-CD96, or anti-CD116, or anti-CD122, or anti-CD152, or anti-CD154 antibody, or a combination thereof.

The antibody can immunodeplete the subject at surprisingly low doses of protein and radiation. As such, this approach avoids certain adverse effects caused by less specific agents like chemotherapeutics, or beam radiation, as the approach targets specific populations of immune cells without affecting stem cells. Moreover, the radioisotopes of the present invention that are selected for labeling the antibodies provides targeted killing of those specific populations of immune cells with minimal adverse effects to surrounding tissues.

Targeted Immunodepletion for Autoimmune Diseases

Because stem cells are continually generating new hematopoietic progenitor cells of both the myeloid and lymphoid compartment of the immune system, removal of the more differentiated cells is temporary, and these depleted cells are eventually replaced by nascently maturing cells derived from the stem cells. As such, immunodepletion could also be used in the case of autoimmune diseases where the differentiated autoreactive cells are depleted and the functional, non-autoreactive immune cells are repopulated from the healthy stem cells.

Accordingly, the present invention also relates to compositions and methods to immunodeplete a subject by administration of a radiolabeled anti-CDXX antibody such as a radiolabeled anti-CD19, or anti-CD20, or anti-CD33, or anti-CD38, or anti-CD45RA, or anti-CD52 antibody, or combination thereof, or a radiolabeled bi-specific antibody that recognized a lymphoid marker and a myeloid marker, to immunodeplete a subject afflicted with an autoimmune disease. Additionally, the present invention relates to a method for reducing the amount of B cells or plasma cells producing pathologic antibodies in the body of a patient suffering from an autoimmune disease (i.e., reducing the numbers of B cells and/or plasma cells in the patient), comprising treating the patient with a therapeutically effective dose of an antibody having specific binding for any one or more of CD19, CD20, CD33, CD38, CD45RA, and CD52.

The term “pathologic antibodies” refers to antibodies exhibiting specific binding against self-antigens, i.e., the antibodies are autoreactive, or those that may deplete differentiated autoreactive cells.

The method can further comprise treating the patient with an additional pharmaceutically active agent, therapeutically effective treatment or other adjunct therapy. The additional pharmaceutically active agent can be a chemotherapeutic agent, complement activation inhibitor, antimetabolite methotrexate), steroid, toleragen, anti-B cell agent, or anticoagulant (heparin, coumadin), antiplatelet agent such as acetylsalicylic acid, TICLID® (ticlopidine HCl), PLAVIX® (clopidogrel bisulfate) or intravenous immunoglobulin. The therapeutically effective treatment includes plasmapheresis or leukapheresis.

Targeted Immunodepletion in Combination with Adoptive Cell Therapy

The immunodepletion may be useful, for example, for improving the outcome of a subsequent therapy wherein the depletion of specific immune cells is desirable, such as the depletion of lymphocytes, i.e., lymphodepletion, prior to a cell-based therapy like CAR T-cell therapy or TCR cell therapy.

According to certain aspects of this method, the subject is afflicted with a malignant hematological disease and is about to undergo adoptive cell therapy to treat the disease (e.g., hematological disease such as multiple myeloma, or malignancy such as a solid tumor). Adoptive cell therapies are known, and include, for example, CAR T-cell therapy (e.g., autologous cell therapy and allogeneic cell therapy). Examples of approved CAR-T cell therapies include, without limitation, KYMRIAH® (tisagenlecleucel) for treating NHL and diffuse large B-cell lymphoma (DLBCL), and YESCARTA® (axicabtagene ciloleucel) for treating NHL.

These presently disclosed methods may improve treatment outcomes for hematological malignancies including solid tumors, and/or may lessen side effects associated with the adoptive cell therapies, such as the CAR-T cell therapies KYMRIAH® and/or YESCARTA®. For example, side effects of adoptive cell therapies include neurotoxicity, cytokine release syndrome (CRS), hypogammaglobulinemia, cytopenias, capillary leak syndrome (CLS), macrophage activation syndrome (MAS), tumor lysis syndrome (TLS), and combinations thereof. Moreover, the presently disclosed methods may prolong persistence of the population of cells expressing the CAR/TCR or the TIL when compared to a method absent administration of the radiolabeled anti-CD45 antibody.

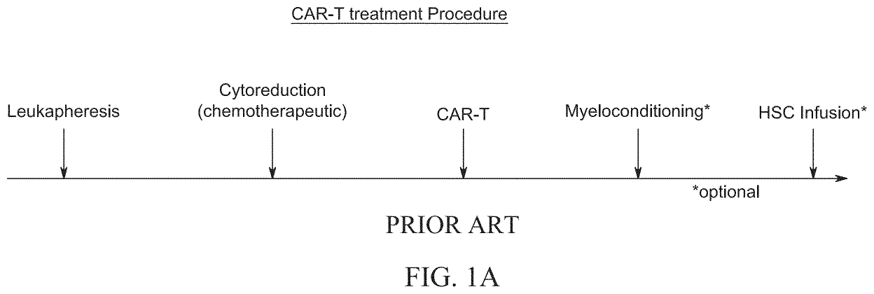

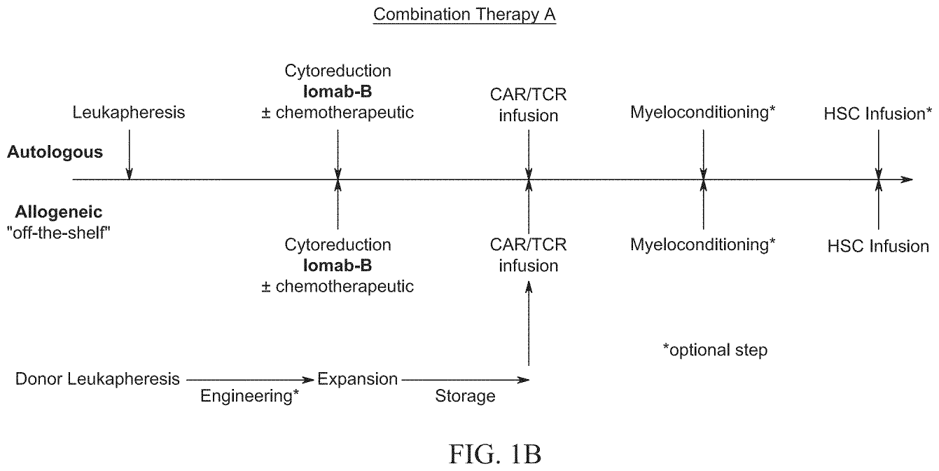

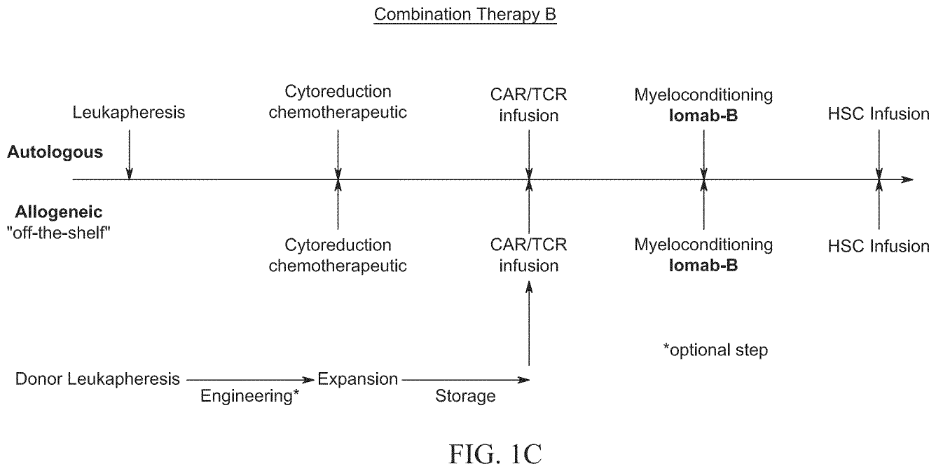

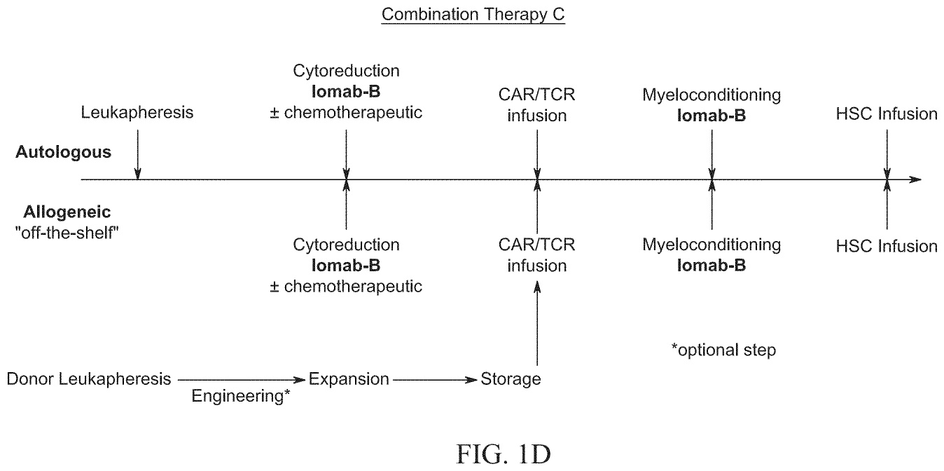

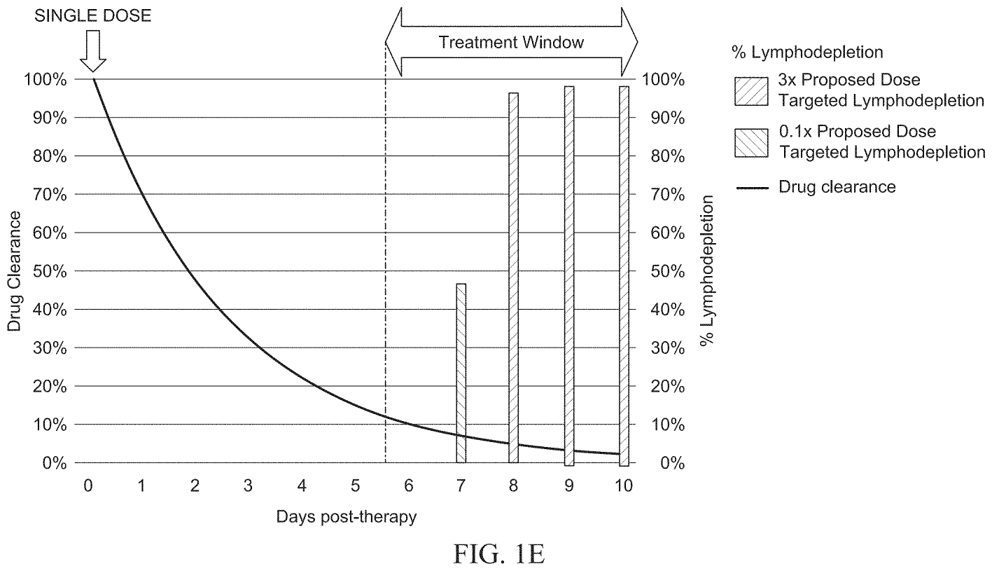

According to certain aspects of the method, the subject is afflicted with cancer (e.g., hematological malignancy or solid cancer) and is about to undergo adoptive cell therapy to treat the cancer. Adoptive cell therapy is known, and includes, for example, CAR T-cell therapy (e.g., autologous cell therapy and allogeneic cell therapy). Adoptive cell therapies provide a method of promoting regression of a cancer in a subject, and generally comprise (i) collecting autologous T-cells (leukapheresis); (ii) expanding the T-cells (culturing); (iii) administering to the subject non-myeloablative lymphodepleting chemotherapy; and (iv) after administering non-myeloablative lymphodepleting chemotherapy, administering to the subject the expanded T-cells (see FIG. 1A).

The methods of the presently disclosed invention include using a radiolabeled antibody in lieu of the lymphodepleting chemotherapy (FIG. 1B), after administration of the expanded cells (e.g., T-cell, NK-cells, dendritic cells, etc.) (FIG. 1C), or both before administration of the expanded cells to lymphodeplete and after administration of the expanded cells (FIG. 1D). The later administration of the radiolabeled antibody (i.e., after administration of the expanded cells) may be used in preparation for transplantation of autologous stem cells (HSCT), or administration of a second effective amount of expanded cells.

Accordingly, the present invention provides methods for the treatment of a proliferative disease, such as a hematological malignancy or a solid cancer, which include administration of a radiolabeled antibody and an adoptive cell therapy. The adoptive cell therapy may generally include apheresis of autologous cells which may be gene edited prior to reinfusion (adoptive cell therapy such as CAR T-cell therapy) after lymphodepletion by the radiolabeled anti-CD45 antibody. Alternatively, allogeneic cells may be reinfused after lymphodepletion to provide the adoptive cell therapy. According to methods of the present invention, the radiolabeled antibody may be provided as a single dose 3 to 9 days, such as 6 to 8 days, prior to the adoptive cell therapy, as shown in FIG. 1E.

Thus, the present invention provides a method for treating a subject afflicted with cancer comprising (i) administering to the subject an amount of a radiolabeled antibody effective to deplete the subject's lymphocytes, and (ii) after a suitable time period, performing adoptive cell therapy on the subject to treat the subject's cancer. Preferably, the subject is human.

Targeted Immunodepletion of Immune Suppressor Cells

The present invention further provides methods for targeted immunodepletion of immune suppressor cells such as regulatory T (T-reg) cells and myeloid-derived suppressor cells (MDSC). Both cells types (i.e., T-regs and MDSC) can dampen the activation and efficacy of CAR-T cell therapies. Moreover, the present invention also provides methods for targeted lymphodepletion of immune suppressor cells such as monocytes and tumor-associated macrophages (TAMs) that have been implicated in cytokine release contributing to toxicities such as cytokine release syndrome (CRS) and neurotoxicity associated with CAR T-cells.

Tumors, both solid and liquid have evolved methods to hijack and/or evade the immune system as a means to perpetuate and thrive. This has been called the hostile tumor immune microenvironment (TME). A classical and relevant example is the up-regulated expression of the ligand PD-L1 on the tumor cell surface to bind PD1 on the surface of T cells, leading to down-modulation of immune cell activation. Interestingly, although blockade of this mechanism has led to remarkable response rates and durable survival in several different types of cancer, most patients do not respond to this form of therapy (i.e., anti-PD1/PD-L1), implying that immune evasion in the tumor microenvironment is multi-faceted and complex. To this end, the tumor, in part through oncogenic expression, signaling, and cytokine production, can confer challenges on the immune system, hindering the mounting of an effective anti-tumor response. This can lead to an environment characterized by oxidative stress, nutrient depletion, an acidic pH, and hypoxia. Further, the presence of these suppressive immune cells (T-regs and MDSC), and tumor-associated macrophages (TAM) can effectively blunt immune cell activation through direct contact or release of suppressive soluble factors and cytokines.

While a patient's endogenous immune system may encounter such an environment and lead to a compromised anti-tumor immune response, adoptive cell therapies such as CAR T-cell therapy may also be susceptible to these immune suppressive mechanisms, restricting the ability of these novel cell therapies to mount an effective response to the tumor.

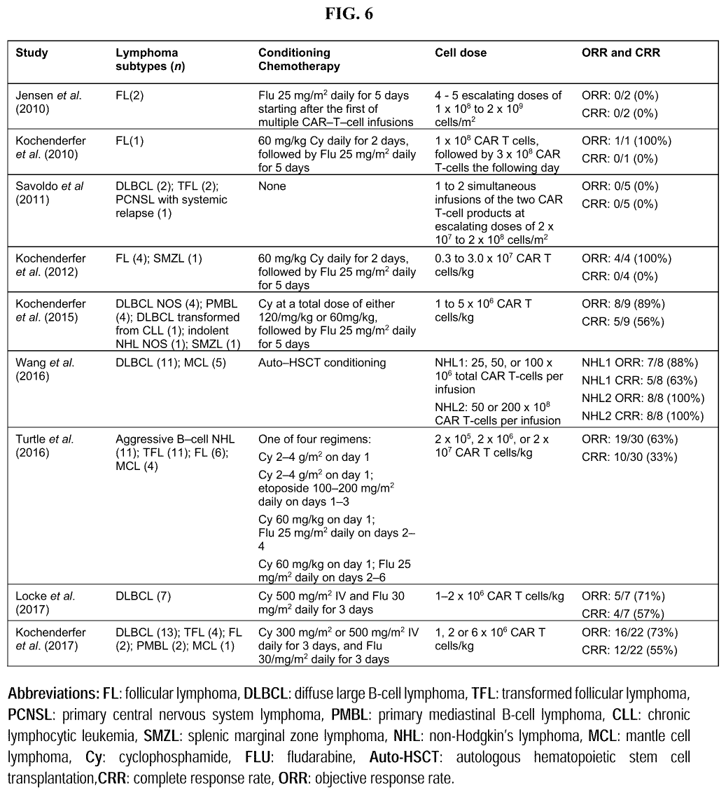

CAR T therapies, and adoptive cell therapy in general, represents one of the most promising anti-cancer strategies emerging from clinical research. Response rates have been extraordinary, on the range of 80% across these tumors, although durable responses have only ranged around 40-50% (see, for example, studies listed in FIG. 6). Nevertheless, these results represent a significant improvement in outcomes for these patients. It is unclear why some patients respond, and others do not, though the tumor immune microenvironment is a likely contributor to modulate the response to cell therapy.

To this end, preclinical and clinical studies have shown that regulatory T cells (T-regs) have an impact on the response to adoptive cell therapy in mice and in patients suffering from melanoma (Gattinoni, et al., 2005, JEM, 202:907; Yao, et al., 2012, Blood, 119:5688). In these studies, depletion of T-regs, whether by intentional depletion or via conditioning with external beam radiation, had a favorable impact on the anti-tumor response to adoptive cell therapy. Interestingly, these and other studies suggest that T-reg depletion is more sustained following treatment with radiation as opposed to chemotherapy-induced conditioning, where a rapid rebound of T-regs was seen with the latter chemotherapy conditioning and poorer outcomes.

MDSCs and TAMs are other cell types implicated in creating a poor tumor immune microenvironment. Through upregulation of metabolic gene expression, such as Indoleamine 2,3-dioxygenase (IDO), adenosine A2A receptor, and CD73, tumors can effectively create a nutrient deprivation in the tumor environment which can blunt T-cell activation. For example, tryptophan metabolism by IDO from tumors and MDSCs leads to T-cell anergy and death, as well as T-reg accumulation at the tumor site. Further, these immune suppressive cells may secrete immune modulatory cytokines such as TGF-β which can also exert a negative effect on T-cell activation.

The negative impact of the hostile tumor immune microenvironment may exist for both liquid and solid tumors, though may be even more pronounced in solid tumors. To this end, early clinical results suggest that the robust response to CAR-T therapy in liquid tumors such as lymphoma, has not been observed in solid tumors, suggesting that factors or conditions exist in solid tumors that may present physical or metabolic barriers to mounting an effective CAR-T-mediated immune response (Newick, et al., 2016, Mol. Ther.—Oncolytics, 3:16006; D'Aloia, et al., 2018, Cell Death and Disease. 9:282-293).

The tumor immune microenvironment has also been implicated in the two primary adverse events associated with CAR-T administration, namely cytokine release syndrome (CRS) and neurotoxicity. Recent preclinical studies have shown that cytokine release leading to CRS or neurotoxicity is due to activated macrophages following recruitment to the site of CAR-T and tumor cells. Mouse study result (Giavadris, et al., 2018, Nat. Med., 24:731) documented that macrophages secrete IL-1 or IL-6 following recruitment and activation by CAR-T cells at the tumor site.

Conditioning has been shown to improve the immune homeostatic environment to enable successful adoptive cell therapy or CAR-T engraftment and expansion in vivo following infusion. However, the use of cytotoxic non-specific chemotherapy can elicit off-target toxicities and has been identified as a risk factor in CRS and neurotoxicity following CAR-T administration. Interestingly, most CAR-T programs exploit the use of the combination of fludarabine and cyclophosphamide (flu/cy) as a conditioning regimen prior to CAR-T. These drugs are often administered 2-7 days prior to adoptive cell therapy infusion, using 2-5-day course of therapy.

The targeted therapy for conditioning of the present invention offers a much-improved strategy for enhancing outcomes with CAR-T. In the invention described herein, not only may lymphocytes be targeted for depletion, but also those immune cell types implicated in mediating a hostile tumor immune microenvironment, and those implicated in CAR-T adverse events such as CRS and neurotoxicity. The present invention targets normal immune cells including T-regs, MDSCs, TAMs, and activated macrophages secreting IL-1 and/or IL-6. In doing so, the inventive protocols and methods may provide a dramatic improvement in CAR-T outcomes and safety.

Furthermore, the invention targets, primarily in hematopoietic tumors, patient cancer cells to reduce tumor burden and increase the probability of CAR-T anti-tumor response. More specifically, the invention provides a therapeutic strategy targeting the CD19, CD20, CD33, CD38, CD45RA, and/or CD52 antigen(s). For example, CD45 is also expressed on most lymphoid and leukemic tumor cells. The radiolabeled anti-CD45 antibody of the present invention will affect a pronounced and sustained suppression of immune cells implicated in modulating CAR-T responses. In this way, the radiation is targeted and impactful on the CD45 cell populations while sparing normal tissues. More specifically, the radiolabeled anti-CD45 antibody may be provided as a single dose at a level sufficiently effective to deplete circulating immune cells within the spleen, lymph nodes, and peripheral blood, but limited in impact on hematopoietic stem cells in the bone marrow. Importantly, in addition to lymphocyte depletion, macrophages, MDSCs and T-regs will be depleted to improve the activation and response to CAR-T therapy and mitigate adverse events CRS and neurotoxicity.

The Radiolabeled Antibodies

The anti-CDXX antibody of the presently disclosed invention may be administered intravenously, intramuscularly, or subcutaneously to a patient. Exemplary administration amounts and rates for the compositions may be as substantially described in WO 2016/187514, incorporated by reference herein.

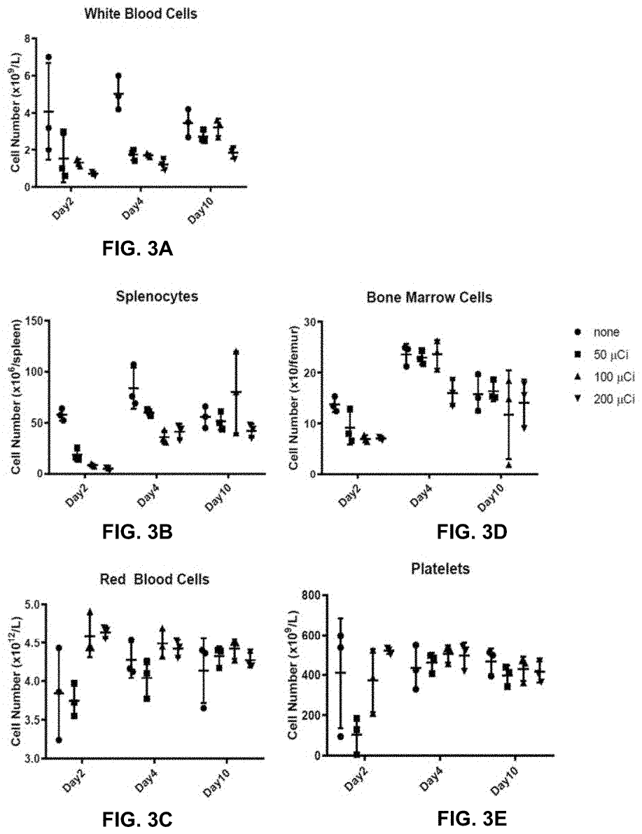

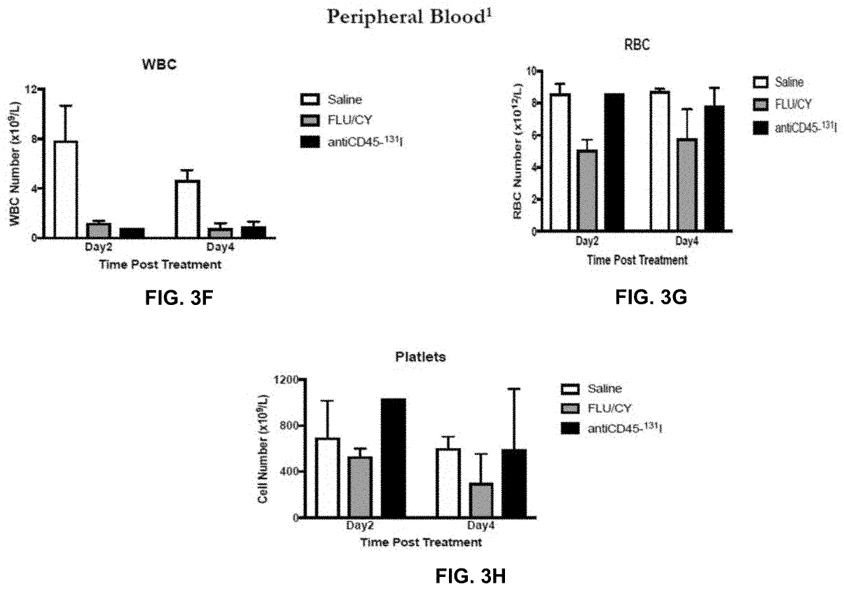

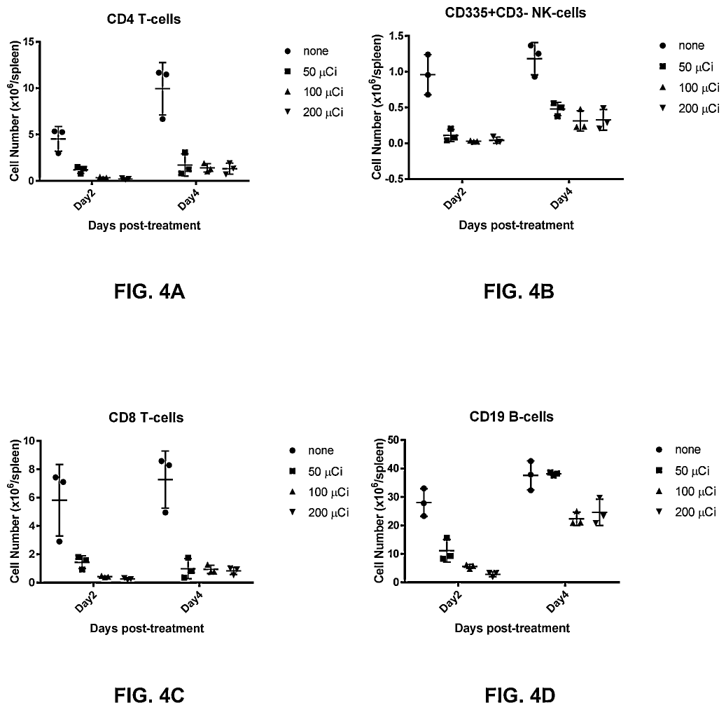

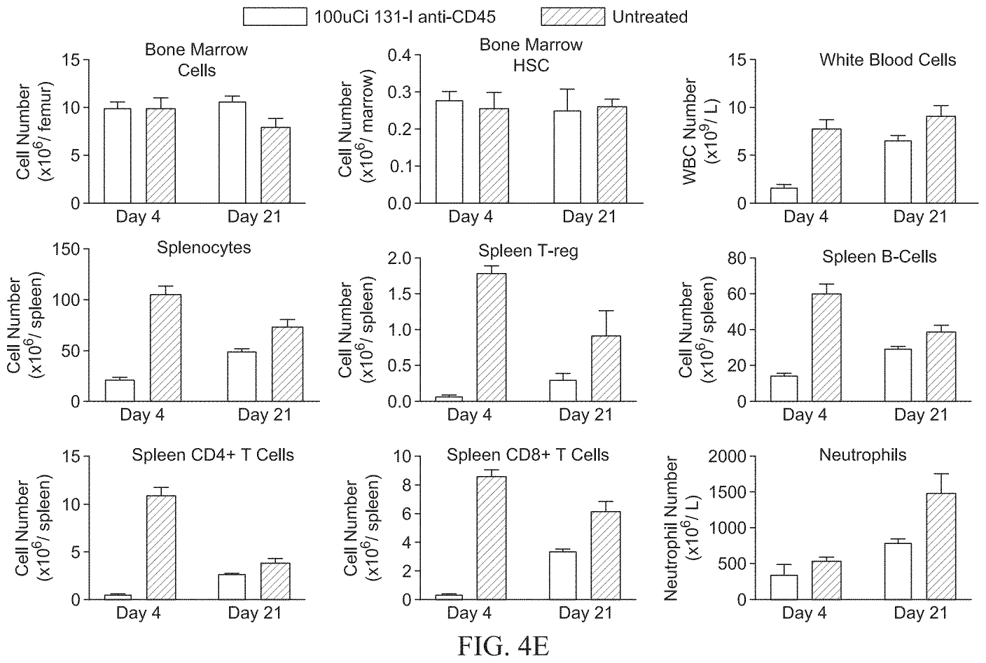

Doses considered effective in safe depletion of circulating immune cells would be doses that deliver 2 Gy or less to the bone marrow, thereby reducing the negative impact on hematopoietic stem cells. Such doses should deplete lymphocytes, immune cells implicated in the hostile immune tumor microenvironment, and tumor cells all leading to enhanced response to ACT or CAR-T therapy. As shown in Table 1, calculations from dosimetry performed in patients receiving ¹³¹I-anti-CD45 (¹³¹I-BC8) indicate that doses below 100 mCi will result in the delivery of a targeted radiation dose to bone marrow, the dose limiting organ, in the range of 200 cGy (2 Gy). Such doses are also found to deliver a higher amount of radiation to the spleen, the site of lymphocytes and myeloid cells for targeted lymphodepletion (see Table 2 and FIGS. 3A-3H and 4A-4E).

According to certain aspects of this method, the radiolabeled anti-CDXX antibody envisioned in this invention is labeled with, without limitation, ¹³¹I, ¹²⁵I, ¹²³I, ⁹⁰Y, ¹⁷⁷Lu, ¹⁸⁶Re, ¹⁸⁸Re, ⁸⁹Sr, ¹⁵³Sm, ³²P, ²²⁵Ac, ²¹³Bi, ²¹³Po, ²¹¹At, ²¹²Bi, ²¹³Bi, ²²³Ra, ²²⁷Th, ¹⁴⁹Tb, ¹³¹I, ¹³⁷Cs, ²¹²Pb, and ¹⁰³Pd, or any combination thereof. Preferably, the radiolabel ¹³¹I or ²²⁵Ac. The antibody may be labeled using methods as detailed in International Publication No. WO 2017/155937.

According to certain aspects of this method, the effective amount of ¹³¹I-antibody is from 10 mCi to 200 mCi. Examples of effective amounts include, without limitation, from 50 mCi to 100 mCi, from 50 mCi to 150 mCi, from 50 mCi to 200 mCi, from 60 mCi to 140 mCi, from 70 mCi to 130 mCi, from 80 mCi to 120 mCi, from 90 mCi to 110 mCi, from 100 mCi to 150 mCi, 50 mCi, 60 mCi, 70 mCi, 80 mCi, 90 mCi, 100 mCi, 110 mCi, 120 mCi, 130 mCi, 140 mCi, 150 mCi, or 200 mCi. According to certain aspects, when the antibody is ¹³¹I labeled, the effective amount is from 10 mCi to 120 mCi, from 20 mCi to 110 mCi, from 25 mCi to 100 mCi, from 30 mCi to 100 mCi, from 40 mCi to 100 mCi, from 50 mCi to 100 mCi, or from 75 mCi to 100 mCi. These low lymphodepletive doses of ¹³¹I are surprising over the known myeloablative doses of ¹³¹I, such as 300 mCi to 1,200 mCi. For example, it was unexpected that these lower doses would yield a drop-in lymphocyte levels. Moreover, these lower doses permit the patient to go home immediately after the ¹³¹I is administered. This would not be possible for a patient receiving, say, a 1,200 mCi dose due to the radiation risk posed to others in close physical proximity to the patient.

According to certain aspects of this method, the effective amount of ²²⁵Ac-antibody is from 0.05 μCi/kg to 5.0 μCi/kg of subject's body weight. Examples of effective amounts include, without limitation, from 0.05 μCi/kg to 5.0 μCi/kg, such as from 0.1 μCi/kg to 0.2 μCi/kg, from 0.2 μCi/kg to 0.3 μCi/kg, from 0.3 μCi/kg to 0.4 μCi/kg, from 0.4 μCi/kg to 0.5 μCi/kg, from 0.5 μCi/kg to 0.6 μCi/kg, from 0.6 μCi/kg to 0.7 μCi/kg, from 0.7 μCi/kg to 0.8 μCi/kg, from 0.8 μCi/kg to 0.9 μCi/kg, from 0.9 μCi/kg to 1.0 μCi/kg, from 1.0 μCi/kg to 1.5 μCi/kg, from 1.5 μCi/kg to 2.0 μCi/kg, from 2.0 μCi/kg to 2.5 μCi/kg, from 2.5 μCi/kg to 3.0 μCi/kg, from 3.0 μCi/kg to 3.5 μCi/kg, from 3.5 μCi/kg to 4.0 μCi/kg, from 4.0 μCi/kg to 4.5 μCi/kg, or from 4.5 μCi/kg to 5.0 μCi/kg.

The effective amount of the radiolabeled antibody may be provided as a single dose. A majority of the antibody administered to a subject typically consists of non-labeled antibody, with the minority being the labeled antibody. The ratio of labeled to non-labeled antibody can be adjusted using known methods. Thus, accordingly to certain aspects of the present invention, the antibody may be provided in a total protein amount of up to 100 mg, such as less than 60 mg, or from 5 mg to 45 mg, or a total protein amount of between 0.1 ug/kg to 1 mg/kg patient weight, such as 1 ug/kg to 1 mg/kg patient weight, or 10 ug/kg to 1 mg/kg patient weight, or 100 ug/kg to 1 mg/kg patient weight, or 0.1 ug/kg to 100 ug/kg patient weight, or 0.1 ug/kg to 50 ug/kg patient weight, or 0.1 ug/kg to 10 ug/kg patient weight, or 0.1 ug/kg to 40 ug/kg patient weight, or 1 ug/kg to 40 ug/kg patient weight, or 0.1 mg/kg to 1.0 mg/kg patient weight, such as from 0.2 mg/kg patient weight to 0.6 mg/kg patient weight.

According to certain aspects of the present invention, the radiolabeled antibody may comprise a labeled fraction and an unlabeled fraction, wherein the ratio of labeled:unlabeled may be from about 0.01:10 to 1:1, such as 0.1:10 to 1:1 labeled:unlabeled.

Moreover, the radiolabeled antibody may be provided as a single dose composition tailored to a specific patient, i.e., as a patient specific therapeutic composition, wherein the amount of labeled and unlabeled antibody in the composition may depend on at least a patient weight, height, body surface area, age, gender, and/or disease state or health status. As such, a total volume of the patient specific therapeutic composition may be provided in a vial that is configured to be wholly administered to the patient in one treatment session, such that little to no composition remains in the vial after administration.

Adoptive Cell Therapy

Adoptive cell therapies are a potent approach for treating cancer but also for treating other diseases such as infections and graft versus host disease. Adoptive cell therapy is the passive transfer of ex vivo grown cells, most commonly immune-derived cells, into a host with the goal of transferring the immunologic functionality and characteristics of the transplant.

Adoptive cell therapies can be autologous (e.g., isolated by leukapheresis, transduced and selected approximately 4 weeks immediately prior to administration), as is common in adoptive T-cell therapies, or allogeneic as typical for treatment of infections or graft-versus-host disease. Moreover, the ACT may be xenogeneic.

Adoptive cell therapies may also comprise transfer of autologous tumor infiltrating lymphocytes (TILs) which may be used to treat patients with advanced solid tumors such as melanoma and hematologic malignancies.

Adoptive cell therapies may also comprise transfer of allogeneic lymphocytes isolated, prepared, and stored (e.g., frozen) “off-the-shelf” from a healthy donor which may be used to treat patients with advanced solid tumors such as melanoma and hematologic malignancies.

The adoptive cell therapies may use cell types such as T-cells, natural killer (NK) cells, delta-gamma T-cells, regulatory T-cells, dendritic cells, and peripheral blood mononuclear cells. The adoptive cell therapies may use monocytes with the purpose of inducing differentiation to dendritic cells subsequent to contact with tumor antigens. Given that monocytes have a fixed mitotic index, permanent manipulation of the host may be diminished.

According to certain aspects, the adoptive cell therapy may be a CAR T-cell therapy. The CAR T-cell can be engineered to target a tumor antigen of interest by way of engineering a desired antigen binding domain that specifically binds to an antigen on a tumor cell. In the context of the present invention, “tumor antigen” or “proliferative disorder antigen” or “antigen associated with a proliferative disorder,” refers to antigens that are common to specific proliferative disorders such as cancer. The antigens discussed herein are merely included by way of example and are not intended to be exclusive, and further examples will be readily apparent to those of skill in the art.

According to certain aspects, the CAR T-cell therapy employs CAR T-cells that target CD19, CD20, CD22, CD30, CD33, CD38, CD123, CD138, CS-1, B-cell maturation antigen (BCMA), MAGEA3, MAGEA3/A6, KRAS, CLL1, MUC-1, HER2, EpCam, GD2, GPC3, GPA7, PSCA, EGFR, EGFRvIII, ROR1, mesothelin, CD33/IL3Ra, c-Met, CD37, PSMA, Glycolipid F77, GD-2, gp100, NY-ESO-1 TCR, FRalpha, CD24, CD44, CD133, CD166, CA-125, HE4, Oval, estrogen receptor, progesterone receptor, uPA, PAI-1, MICA, MICB, ULBP1, ULBP2, ULBP3, ULBP4, ULBP5 or ULBP6, or a combination thereof (e.g., both CD33 and CD123). It is envisioned in this invention that, according to certain aspects, the subject afflicted with cancer is a patient with a higher burden of disease (≥5% bone marrow blasts) with a greater incidence of adverse events such as cytokine release syndrome and shorter long-term survival after CAR T.

The CAR T-cell may comprise an antigen binding domain capable of targeting two or more different antigens (i.e., bi-specific or bivalent, tri-specific or trivalent, tetra-specific, etc.). As such, the CAR T-cell may comprise a first antigen binding domain that binds to a first antigen and a second antigen binding domain that binds to a second antigen (e.g., tandem CAR). For example, the CAR T-cell may comprise a CD19 binding domain and a CD22 binding domain and may thus recognize and bind to both CD19 and CD22. Or further, the CAR T-cell may comprise a CD19 binding domain and a CD20 binding domain and may thus recognize and bind to both CD19 and CD20.

Alternatively, each cell in the population of cells, or the overall population of cells, may comprise more than one distinct CAR T-cell (e.g., construct), wherein each CAR T-cell construct may recognize a different antigen. For example, the population of CAR T-cells may target three antigens such as, for example, HER2, IL13Ra2, and EphA2.

According to certain aspects of the present invention, the population of cells, whether autologous or allogeneic, may be engineered using gene editing technology such as by CRISPR/cas9 (clustered regularly interspaced short palindromic repeats/CRISPR associated protein 9), Zinc Finger Nucleases (ZFN), or transcription activator-like effector nuclease (TALEN). These technologies, recognized and practiced in the art of genetic engineering, enable selective editing, disruption, or insertion of targeted sequences to modify the genome of the cell of interest. Accordingly, isolated autologous or allogeneic cells for adoptive transfer practiced in the current invention may be edited to delete or replace a known gene or sequence. For example, the T cell receptor (TCR) in an allogeneic T cell population may be deleted or replaced prior to or after CAR-T transduction as a means to eliminate graft-versus-host disease in recipient patients.

According to certain aspects of the present invention, the population of cells may comprise a population of T-cells, NK-cells, or dendritic cells expressing a CAR, wherein the CAR comprises an extracellular antibody or antibody fragment that includes a humanized anti-CD19 binding domain or a humanized anti-BCMA binding domain, a transmembrane domain, and one or more cytoplasmic co-stimulatory signaling domains. The population of cells may comprise a population of cells expressing a CAR, wherein the CAR comprises an extracellular antibody or antibody fragment that includes two or more binding domains, such as a humanized anti-CD19 binding domain, a humanized anti-CD22 binding domain, and/or a humanized anti-BCMA binding domain, and a transmembrane domain and one or more cytoplasmic co-stimulatory signaling domains.

CAR cell therapy has shown unprecedented initial response rates in advanced B-cell malignancies; however, relapse after CAR cell infusion, and limited therapeutic success in solid tumors is a major hurdle in successful CAR regimens. This latter limitation is mainly attributable to the hostile microenvironment of a solid tumor. Anatomical barriers such as the tumor stroma, and immunosuppressive cytokines and immune cells which are harmful to the infiltration of infused CAR modified cells into tumor sites, both limit the success of CAR cell therapy. Armored CAR may be used to circumvent certain of these limitations. These CAR cells are further modified to express immune-modulatory proteins, such as cytokines (e.g., IL-2, IL-12 or IL-15), which may stimulate T-cell activation and recruitment, and may thus aid in combating the tumor microenvironment. Thus, according to certain aspects of the present invention, the population of cells may comprise a population of cells expressing a CAR and further expressing an immune modulatory protein such as, for example, IL-2, IL-12, or IL-15.

An adoptive cell therapy of the present invention includes a population of cells expressing T-cell receptors (TCRs). TCRs are antigen-specific molecules that are responsible for recognizing antigenic peptides presented in the context of a product of the major histocompatibility complex (MHC) on the surface of antigen presenting cells or any nucleated cell (e.g., all human cells in the body, except red blood cells). In contrast, antibodies typically recognize soluble or cell-surface antigens, and do not require presentation of the antigen by an MHC. This system endows T-cells, via their TCRs, with the potential ability to recognize the entire array of intracellular antigens expressed by a cell (including virus proteins) that are processed intracellularly into short peptides, bound to an intracellular MHC molecule, and delivered to the surface as a peptide-MHC complex. This system allows virtually any foreign protein (e.g., mutated cancer antigen or virus protein) or aberrantly expressed protein to serve as a target for T-cells.

As indicated above, on-target, off-tissue adverse events, typical of a cytokine release syndrome (CRS), have been observed for adoptive cell therapy. To maintain the benefit of these revolutionary treatments while minimizing the risk, a tunable safety switch may be incorporated which may control the activity level of CAR-expressing or TCR-expressing cells. That is, an inducible costimulatory chimeric polypeptide may be included which may allow for a sustained, modulated control of the CAR or TCR. The ligand inducer may activate the CAR-expressing or TCR-expressing cell by multimerizing an inducible chimeric signaling molecule for example, which may induce intracellular signaling pathways, leading to the activation of the target cells (T-cell, NK-cell, TIL, dendritic cell). In the absence of the ligand inducer, the target cell is quiescent, or has a basal level of activity.

Thus, according to certain aspects of the present invention, a switch may be added which activates the CAR or TCR (i.e., safety switch CAR or TCR, goCAR or goTCR). The CAR or TCR expressing cell may be designed to only be fully activated when exposed to both a cancer cell (e.g., target antigen) and a chemical agent (e.g., rimiducid, rapamycin), thus providing a means to control the degree of activation of the CAR or TCR cells by adjusting the administration schedule of the chemical agent. The CAR or TCR would still work in a target manner, but on a controllable schedule which may reduce certain of the side effects of adoptive cell therapy. Thus, according to certain aspects of the present invention, the ACT may include goCAR or goTCR cell therapies.

According to certain aspects of the present invention, the engineered CAR cell may be allogeneic from a healthy donor and be further engineered to ablate or replace the endogenous TCR by gene editing technology such as CRISPR/cas9, ZFN, or TALEN, wherein the deletion of the endogenous TCR serves to eliminate CAR driven graft-versus-host disease.

According to certain aspects of the present invention, autologous cells (e.g., T-cell or NK-cells or dendritic cells) may be collected from the subject. These cells may be obtained from a number of sources, including peripheral blood mononuclear cells, bone marrow, lymph node tissue, cord blood, thymus tissue, tissue from a site of infection, ascites, pleural effusion, spleen tissue, and tumors. According to certain aspects of the present invention, allogeneic or xenogeneic cells may be used, typically isolated from healthy donors. When the T-cells, NK cells, dendritic cells, or pluripotent stem cells are allogeneic or xenogeneic cells, any number of cell lines available in the art may be used.

The cells can be obtained from a unit of blood collected from a subject using any number of techniques known to the skilled artisan, such as Ficoll™ separation. According to certain aspects of the present invention, cells from the circulating blood of an individual may be obtained by apheresis. The apheresis product typically contains lymphocytes, including T-cells, monocytes, granulocytes, B-cells, other nucleated white blood cells, red blood cells, and platelets.

Enrichment of a cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. According to certain aspects of the present invention, it may be desirable to enrich for or positively select for a cell population. For example, positive enrichment for a regulatory T-cell may use positive selection for CD4+, CD25+, CD62Lhi, GITR+, and FoxP3+.

The collected cells may be engineered to express the CAR or TCR by any of a number of methods known in the art. Moreover, the engineered cells may be expanded by any of a number of methods known in the art. As detailed above, the CAR or TCR may be bi-specific, tri-specific, or quadra-specific; the CAR or TCR may include a switch such as a goCAR or goTCR, or a safety switch CAR or TCR; the CAR or TCR may express immune-modulatory proteins such as an armored CAR or TCR.

Additional agents which are known to suppress bone marrow, or may act to lymphodeplete, or alter the microenvironment of a tumor, may be used in combination with the radiolabeled antibodies indicated above. For example, gemcitabine may deplete myeloid derived suppressor cells and/or bone marrow, while PD1, PD-L1, TIM3 or LAG-3 antagonist antibodies, GITR or OX40 co-stimulatory antibodies, IDO inhibitors, A2aR antagonists, or CD73 antagonists, may alter or shift the tumor microenvironment. When the additional agent is an antibody, it may be provided as a separate agent or composition, or as one arm of a bi-specific antibody in combination with any of the antibody targets listed herein (i.e., CD19, CD20, CD33, CD38, CD45RA, CD52).

According to certain aspects of the present invention, the collection of blood samples or apheresis product from a subject may be at any time period prior to when the expanded cells as described herein might be needed. As such, the source of the cells to be engineered and expanded (or simply expanded in the case of TILs) can be collected at any time point necessary, and desired cells, such as T-cells, NK-cells, dendritic cells, or TILs, can be isolated and frozen for later use in adoptive cell therapy, such as those adoptive cell therapies described herein.

Thus, according to certain aspects of the present invention, the blood sample or apheresis may be from a generally healthy subject, or a generally healthy subject who is at risk of developing a disease, but who has not yet developed a disease, and the cells of interest may be isolated and frozen for later use. The cells may be expanded, frozen, and used at a later time. In certain cases, the cell samples may be collected from a subject shortly after diagnosis of a particular disease as described herein but prior to any treatments.

According to certain aspects of the present invention, the cells may be isolated from a subject and used fresh, or frozen for later use, in conjunction with (e.g., before, simultaneously or following) lymphodepletion using any of the radiolabeled antibody of the present invention.

According to certain aspects of the present invention, the population of cells expressing the CAR/TCR may be administered to the subject by dose fractionation, wherein a first percentage of a total dose is administered on a first day of treatment, a second percentage of the total dose is administered on a subsequent day of treatment, and optionally, a third percentage of the total dose is administered on a yet subsequent day of treatment.

An exemplary total dose comprises 10³ to 10¹¹ cells/kg body weight of the subject, such as 10³ to 10¹⁰ cells/kg body weight, or 10³ to 10⁹ cells/kg body weight of the subject, or 10³ to 10⁸ cells/kg body weight of the subject, or 10³ to 10⁷ cells/kg body weight of the subject, or 10³ to 10⁶ cells/kg body weight of the subject, or 10³ to 10⁵ cells/kg body weight of the subject. Moreover, an exemplary total dose comprises 10⁴ to 10¹¹ cells/kg body weight of the subject, such as 10⁵ to 10¹¹ cells/kg body weight, or 10⁶ to 10¹¹ cells/kg body weight of the subject, or 10⁷ to 10¹¹ cells/kg body weight of the subject.

An exemplary total dose may be administered based on a patient body surface area rather than the body weight. As such, the total dose may include 10³ to 10¹³ cells per m².

An exemplary dose may be based on a flat or fixed dosing schedule rather than on body weight or body surface area. Flat-fixed dosing may avoid potential dose calculation mistakes. Additionally, genotyping and phenotyping strategies, and therapeutic drug monitoring, may be used to calculate the proper dose. That is, dosing may be based on a patient's immune repertoire of immunosuppressive cells (e.g., T-regs, MDSC), and/or disease burden. As such, the total dose may include 10³ to 10¹³ total cells.

According to certain aspects of the present invention, cells may be obtained from a subject directly following a treatment. In this regard, it has been observed that following certain cancer treatments, in particular treatments with drugs that damage the immune system, shortly after treatment during the period when subjects would normally be recovering from the treatment, the quality of certain cells (e.g., T-cells) obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T-cells, NK-cells, dendritic cells, or other cells of the hematopoietic lineage, during this recovery phase.

According to certain aspects of the present invention, the radiolabeled antibody may be administered after administration of the effective dose of the population of cells expressing the CAR/TCR, or the TIL. The effective amount of the radiolabeled antibody may be an amount sufficient to induce lymphodepletion in the subject. According to certain aspects, the effective amount of the radiolabeled antibody may be an amount sufficient to induce myeloablation in the subject.

According to certain aspects of the present invention, the radiolabeled antibody may be administered 1 to 3 months after administration of the population of cells expressing the CAR/TCR, or the TIL, and the effective amount of the radiolabeled antibody is an amount sufficient to induce lymphodepletion in the subject. Such may be done in preparation for transplantation of autologous stem cells; or administration of a second effective amount of the population of cells expressing the CAR/TCR.

According to certain aspects of the present invention, the radiolabeled antibody may be administered to the subject both before and after adoptive cell therapy. That is, the method may comprise an apheresis step to collect a population of cells that may engineered to express a CAR/TCR, and expanded, followed by administration of a first dose of an effective amount of a radiolabeled antibody. The population of cells expressing the CAR/TCR may then be administered to the subject, followed by administration of a second dose of an effective amount of a radiolabeled antibody. A second dose of the population of cells expressing the CAR/TCR may then be administered. This second dose of the population of cells expressing the CAR/TCR may be the same as the first dose or may be different. That is, the first and second dose may be doses of the same population of cells expressing the same CAR/TCR. Alternatively, the second dose may be a different effective amount of the same population of cells expressing the same CAR/TCR.

According to certain aspects of the present invention, the second dose may be the same or a different effective amount of a different population of cells expressing the same or a different CAR/TCR. Differences in the CAR/TCR may be in any aspect of the CAR/TCR such as, for example, different binding or antigen recognition domains or co-stimulatory domains. The second dose may additionally or alternatively include secreting cells with IL-12 or may even include adjuvant immunotherapies with small molecule inhibitors such as BTK, P13K, IDO inhibitors either concurrent or sequential to the cell therapy infusion.

Thus, according to certain aspects of the present invention, a second dose of the population of cells expressing the CAR/TCR may include a second population of cells expressing a second CAR/TCR, wherein the second CAR/TCR differs from the CAR/TCR in the first dose. For example, a second dose may comprise a second population of activated T-cells or NK-cells or dendritic cells expressing the second CAR/TCR, wherein the second CAR/TCR may comprise a second CAR having one or more of the extracellular, transmembrane, or cytoplasmic co-stimulatory signaling domains which differ from the CAR in the first dose. As example, the second CAR may recognize a different antigen, or may be expressed on a different cell type, or may include different cytoplasmic co-stimulatory signaling domains, etc.

Administration of the second dose of the radiolabeled antibody after the population of cells expressing the CAR/TCR have been administered may be used in subjects who have not shown a complete response (CR) after administration of the population of cells expressing the CAR/TCR. Moreover, administration of the second dose of the radiolabeled antibody after administration of the population of cells expressing the CAR/TCR may be used when the subject has relapsed or is identified as having relapsed, such as with antigen positive disease (e.g., CD-19+) or antigen negative disease (CD-19-), after administration of the population of cells expressing the CAR/TCR.

According to certain aspects of the present invention, the methods may comprise administration of one or more additional therapeutic agents. Exemplary therapeutic agents include a chemotherapeutic agent, an anti-inflammatory agent, an immunosuppressive, an immunomodulatory agent, or a combination thereof.

Therapeutic agents may be administered according to any standard dose regime known in the field. Exemplary chemotherapeutic agents include anti-mitotic agent, such as taxanes, for instance docetaxel, and paclitaxel, and vinca alkaloids, for instance vindesine, vincristine, vinblastine, and vinorelbine. Exemplary chemotherapeutic agents include a topoisomerase inhibitor, such as topotecan.

Exemplary chemotherapeutic agents include a growth factor inhibitor, a tyrosine kinase inhibitor, a histone deacetylase inhibitor, a P38a MAP kinase inhibitor, inhibitors of angiogenesis, neovascularization, and/or other vascularization, a colony stimulating factor, an erythropoietic agent, an anti-anergic agents, an immunosuppressive and/or immunomodulatory agent, a virus, viral proteins, immune checkpoint inhibitors, BCR inhibitors (e.g., BTK, P13K, etc.), immune-metabolic agents (e.g., IDO, arginase, glutaminase inhibitors, etc.), and the like. According to certain aspects of the present invention, the one or more therapeutic agents may comprise an antimyeloma agent. Exemplary antimyeloma agents include dexamethasone, melphalan, doxorubicin, bortezomib, lenalidomide, prednisone, carmustine, etoposide, cisplatin, vincristine, cyclophosphamide, and thalidomide, several of which are indicated above as chemotherapeutic agents, anti-inflammatory agents, or immunosuppressive agents.

### EXAMPLES

The following examples provide experimental designs and specific results for compositions and methods of the present invention using one of the radiolabeled antibodies according to the present invention: anti-CD45 (monoclonal antibody is BC8).

### Example 1—¹³¹I-BC8 (Iomab-B)

The Iomab-B drug product is a radio-iodinated anti-CD45 murine monoclonal antibody (mAb) (¹³¹I-BC8). It is specific for the hematopoietic CD45 antigen. The Iomab-B drug product is supplied as a sterile formulation contained in a container closure system consisting of depyrogenated Type 1 50 mL glass vial, sterilized grey chlorobutyl rubber stopper, and open top style aluminum seal. Each dose vial also contains a drug product fill volume of 45 mL in a 50 mL vial. Similarly, it is provided as a single use dose for complete infusion during intravenous administration and contains patient-specific radioactivity from 1 mCi to 200 mCi (e.g., 100 mCi or 150 mCi) of ¹³¹I and 6-60 mg of BC8, such as 6 to 45 mg. BC8 antibody dose may be determined according to the ideal body weight at a level of 0.5 mg/kg. The drug product is co-administered in-line with 0.9% Sodium Chloride Injection USP (normal saline solution) to the patients at a ratio of 1:9 of drug product to saline solution. The total drug product and saline infusion volume of approximately 430-450 mL is administered over varied durations, since the infusion rate depends on the amount of BC8 antibody in the 45 mL drug product fill volume.

International Publication No. WO 2017/155937 teaches the full structure of BC8, and methods for making ¹³¹I-BC8.

### Example 2—²²⁵Ac-BC8

Conjugation of Anti-CD45 BC8 with DOTA and Subsequent Labeling with ²²⁵Ac: The antibody BC8 (2 mg) was equilibrated with conjugation buffer (Na carbonate buffer with 1 mM EDTA, pH=8.5-9.0) by four ultrafiltration spins using a Centricon filter with a MW cutoff of 50,000, or a Vivaspin ultrafiltration tube with a MW cutoff of 50,000. 1.5 ml of conjugation buffer per spin was used. For each spin, the antibody was spun for 5-20 minutes, at 53,000 RPM and at 4° C. to a residual retentate volume of 100-200 μl. The antibody was incubated at 4° C. for 30 minutes following the 2^{nd} and 3^{rd} spins to allow for equilibration. For DOTA conjugation, a solution of S-2-(4-Isothiocyanatobenzyl)-1,4,7,10 tetraazacyclododecanetetraacetic acid (p-SCN-Bz-DOTA; MW=687) at 3 mg/ml in 0.15M NH40Ac was prepared by dissolution and vortexing. DOTA-Bz-pSCN and BC8 antibody (at >5 mg/ml) was mixed together at a 7.5 molar ratio (DOTA:antibody) in an Eppendorf tube and incubated for 15 hours at room temperature. For purification of the DOTA-antibody conjugate, unreacted DOTA-Bz-pSCN was removed by seven rounds of ultrafiltration with 1.5 ml of 0.15M NH40Ac buffer, pH=6.5 to a volume of approximately 100 μl. After the final wash, 0.15 M NH40Ac buffer was added to bring the material to a final concentration of approximately 1 mg/ml. The final concentration of the DOTA-BC8 conjugate was measured and the number of DOTA molecules conjugated to the antibody was determined to be 1.2-1.5 DOTA to antibody.

Radiolabeling of DOTA-Antibody Conjugates with ²²⁵Ac: To label the DOTA-BC8 conjugate with ²²⁵Ac, 15 μL 0.15M NH₄OAc buffer, pH=6.5, was mixed with 2 μL (10m) DOTA-BC8 (5 mg/ml) in an Eppendorf reaction tube. Four μL of ²²⁵Ac (10 μCi) in 0.05 M HCl were subsequently added, the contents of the tube were mixed with a pipette tip, and the reaction mixture was incubated at 37° C. for 90 minutes with shaking at 100 rpm. At the end of the incubation period, 3 μL of 1 mM DTPA solution was added to the reaction mixture and incubated at room temperature for 20 minutes to bind un-complexed ²²⁵Ac. Instant thin layer chromatography (ITLC) was performed with a 10 cm silica gel strip and a 10 mM EDTA/normal saline mobile phase to determine the radiochemical purity of ²²⁵Ac-BC8, separating Ac-labeled BC8 from ²²⁵Ac-DTPA and counting sections in a gamma counter equipped with a multichannel analyzer. The radiolabeling efficiency over several runs was determined to be greater than 80%.

### Example 3—Change in Absolute Neutrophil Count

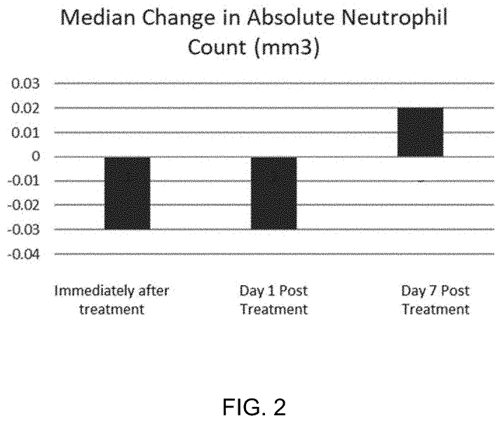

CD45 is a cell surface protein expressed on most immune cell types, including both lymphocytes and neutrophils. Iomab-B (¹³¹I-BC8) radioimmunotherapy targets and delivers its beta-emitting payload to CD45-positive cells. It would have been expected that all CD45-positive cell types would be equally susceptible to depletion following dosing with ¹³¹I-BC8. Data from human clinical trials, shown in FIG. 2, shows the relative absolute neutrophil counts for human subjects at various time points following a 10 mCi dose of ¹³¹I-BC8, presented as fold-increase or decrease. While absolute lymphocyte counts were significantly reduced and exhibited sustained depletion over time, median absolute neutrophil counts exhibited a minimal decrease following ¹³¹I-BC8 dosing, with rapid recovery. This is a surprising finding. The limited impact of ¹³¹I-BC8 on neutrophils and their rapid rebound would be expected to benefit patients by preventing infections that might otherwise occur.

### Example 4—Clinical Data Demonstrating Lymphodepletion and Clearance

Clinical data obtained from patients given low dose levels of ¹³¹I-BC8 show consistent peripheral lymphocyte reduction. Pharmacokinetic data show fast clearance of ¹³¹I-BC8. This clearance limits interaction with CAR T products. Increased disease control and prolonged lymphodepletion allow for a flexible window of time between ¹³¹I-BC8 administration and CAR T infusion, which in turn prevents toxicity. For example, as shown in FIGS. 3A-3H, immune cell analysis following ¹³¹I-anti-CD45 antibody targeted lymphodepletion shows a selective depletion of WBC (FIG. 3A) in peripheral blood and splenic immune cell populations (FIG. 3B), with minimal impact on bone marrow compartment (FIG. 3C) and sparing of RBCs and platelets (FIGS. 3D and 3E, respectively). FIGS. 3F-3H provide bar graphs of similar trial results at days 2 and 4 only. Somewhat surprisingly, the splenic T-reg levels were found to be persistently depressed (depleted) even after 21 days. FIG. 12 shows the rapid clearance of the ¹³¹I-BC8 from the circulating blood in patients. On average, 59 percent of the radiolabeled BC8 antibody cleared from blood with a biological half-time of 0.65 hours (39 minutes), and 41 percent cleared with a biological half-time of 31 hours.

### Example 5—Various CAR-T Cell Therapies in Development, and their Lymphodepletion Regimens

There are numerous CAR-T cell therapies in development, each with its own lymphodepletion regime. The chart in FIG. 6 shows selected published trials of anti-CD19 CAR T-cell therapy for patients with B-cell NHL. Most of these trials employ a chemotherapeutic lymphodepletion regime.

### Example 6—¹³¹I-BC8-Based Lymphodepletion Prior To KYMRIAH®

In this example, the subject invention is used to treat a human subject afflicted with NHL or DLBCL. In the first case, this method comprises (i) administering to an NHL patient from 25 mCi to 200 mCi (e.g., 100 mCi or 150 mCi) of ¹³¹I-BC8, and (ii) after 6, 7 or 8 days, performing KYMRIAH® (tisagenlecleucel) therapy on the patient according to its known protocol. In the second case, this method comprises (i) administering to a DLBCL patient from 25 mCi to 200 mCi (e.g., 100 mCi or 150 mCi) of ¹³¹I-BC8, and (ii) after 6, 7 or 8 days, performing KYMRIAH® therapy on the patient according to its known protocol.

### Example 7—¹³¹I-BC8-Based Lymphodepletion Prior To YESCARTA®

In this example, the subject invention is used to treat a human subject afflicted with NHL. This method comprises (i) administering to an NHL patient from 25 mCi to 200 mCi (e.g., 100 mCi or 150 mCi) of ¹³¹I-BC8, and (ii) after 6, 7 or 8 days, performing YESCARTA® (axicabtagene ciloleucel) therapy on the patient according to its known protocol.

### Example 8—Preclinical Modeling of Anti-CD45 Radioimmunotherapy-Mediated Conditioning/Lymphodepletion

Summary: Prior to a patient receiving a dose of an adoptive cell transfer such as engineered autologous or allogeneic CAR T-cells, it is common to perform a lymphodepletion step often using high dose chemotherapy. This process is considered important to create sufficient space in the immune microenvironment, e.g., bone marrow, to allow the transferred cells to engraft. Further, it appears to elicit a favorable cytokine profile for establishment and proliferation of the donor lymphocytes. Anti-CD45 radioimmunotherapy is being investigated in a Phase III clinical trial as a myeloablative regimen prior to allogeneic hematopoietic cell transplantation in AML patients. Results from this trial suggest that lower doses of ¹³¹I-anti-CD45 radioimmunotherapy may be sufficiently lymphodepleting but not myeloablative.

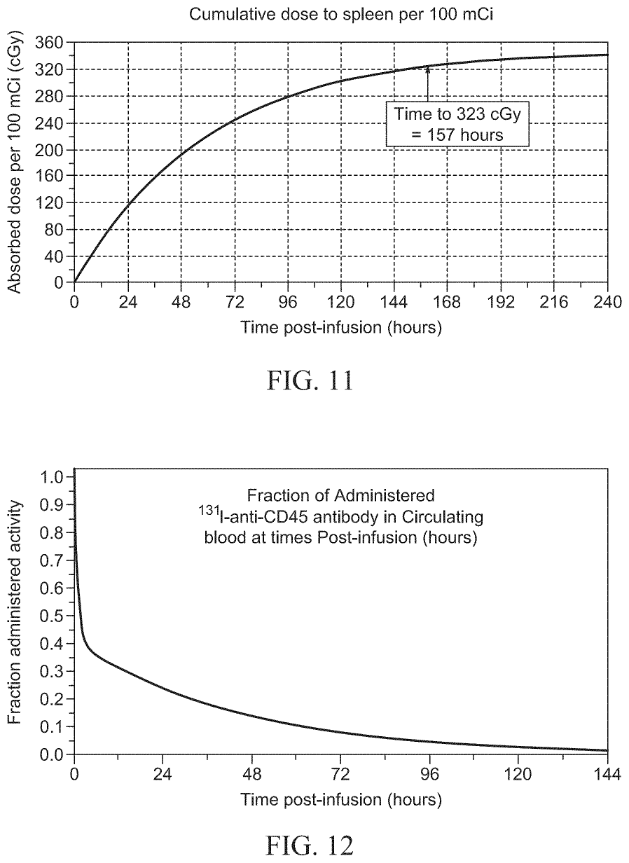

As example, studies investigating the cumulative radiation dose absorbed in the spleens of human patients receiving single doses of 50 mCi to 200 mCi of ¹³¹I-anti-CD45 antibody have been performed. As shown in Table 3, the time at which the remaining absorbed dose to the spleen will not exceed 25cGy varies with the total dose of ¹³¹I-anti-CD45 antibody. Moreover, FIG. 11 show the cumulative dose rate to the spleen for patients administered 100 mCi CD45 antibody through infusion, wherein the dose rate to the spleen is plotted against time-post infusion.

TABLE 3

| | Time to remaining | Days (approximate) | Total spleen |
|---|---|---|---|
| ¹³¹I Activity | absorbed dose less | post-infusion | absorbed |
| Administered | than 25 cGy | (nearest half-day) | dose (cGy) |
| 50 mCi | 117 hours | 4.9 | 174 |
| 75 mCi | 141 hours | 5.9 | 261 |
| 100 mCi | 157 hours | 6.6 | 348 |
| 150 mCi | 182 hours | 7.6 | 522 |
| 200 mCi | 199 hours | 8.3 | 696 |

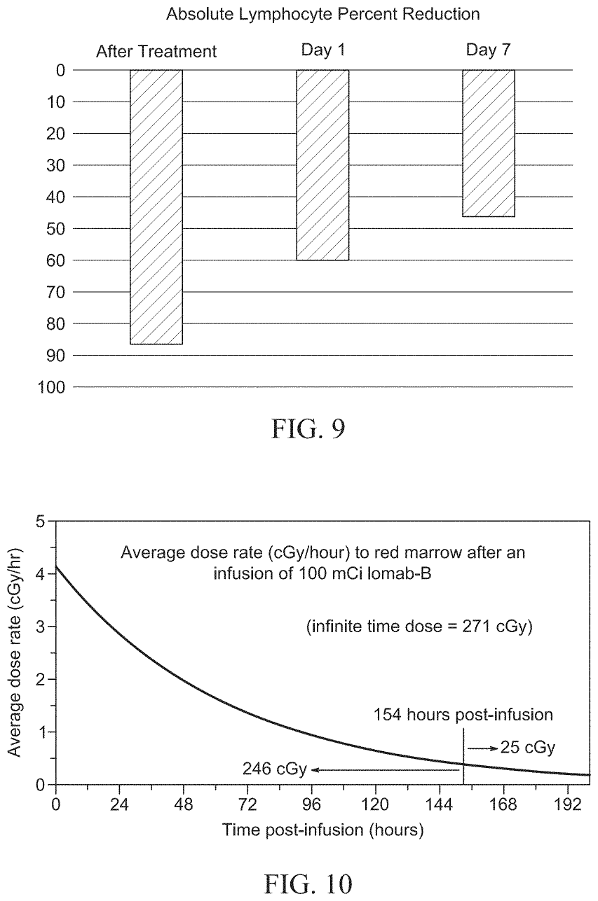

Preclinical studies of the effects of low dose ¹³¹I-anti-CD45 radioimmunotherapy (surrogate 30F11) investigating the lymphodepletive response on particular immune cell types, and not the changes in cytokine expression in response to this form of conditioning, have been performed. For example, FIG. 9 shows transient lymphodepletion in human patients receiving 5 to 20 mCi of ¹³¹I-anti-CD45 antibody (median dose 8.4 mCi for 13 patients). The results of such studies will be supportive in developing and planning human clinical studies utilizing anti-CD45 radioimmunotherapy as a non-myeloablative conditioning regimen prior to the administration of autologous or allogeneic adoptive cell transfer (ACT).

Current studies include immune competent mice (e.g., female C57Bl/6 mice 8 to 12 weeks old) using a CD45 radiolabeled (¹³¹I) anti-mouse receptor antibody to deplete bone marrow and peripheral blood of CD45+ immune cells for modeling of CD45-radioimmunotherapy agents as conditioning regimens for receiving ACT. The comparator will be treatment with the chemotherapy combination of cyclophosphamide (Cy) with fludarabine (Flu). At up to three time points post-treatment, animals will be sacrificed, and peripheral blood, spleen, and bone marrow samples collected for immunophenotyping to evaluate lymphoid and myeloid subsets for lymphodepletion, and blood (serum) collected for cytokine profiling in response to the various treatment regimens.

Materials: Mice (3 per each time point group), e.g., female adolescent C57Bl/6 mice (30 mice total). Mouse surrogate anti-CD45 antibody 30F11 (vendor: Millipore Sigma: MABF321, rat IgG2b). ¹³¹I for labeling. Fludarabine (Flu)+Cyclophosphamide (Cy) (treatment: 250 mg/kg Cy+50 mg/kg Flu)

Part I: Labeling and in vitro characterization of surrogate CD45 antibody 30F11-Perform iodination of 30F11 antibody; perform immunoreactivity to mouse-CD45+ cells (e.g., B6-Ly5a splenocytes for 1 hour at 5 ng/ml: target >70% immunoreactivity)

Part II: (a) (i) Prepare dose for in vivo study (0.5 mCi in 100 ug administered in 200 ul; per Matthews, et al., 2001, Blood, 2:737-745) (ii) Matthews dosimetry results: 0.5 mCi dose: 54 Gy to spleen, 17 Gy to marrow, 8 Gy to lung, and 5 Gy to liver. In other studies, 0.05, 0.1 and 0.2 mCi in 100 ug were administered for determination of dose response.

(b) The radioimmunotherapy regimen will be administered IP. Nine mice per group will receive CD45-radioimmunotherapy or chemotherapy (Cy/Flu) combination. Twelve mice will serve as pre-treatment and no treatment controls.

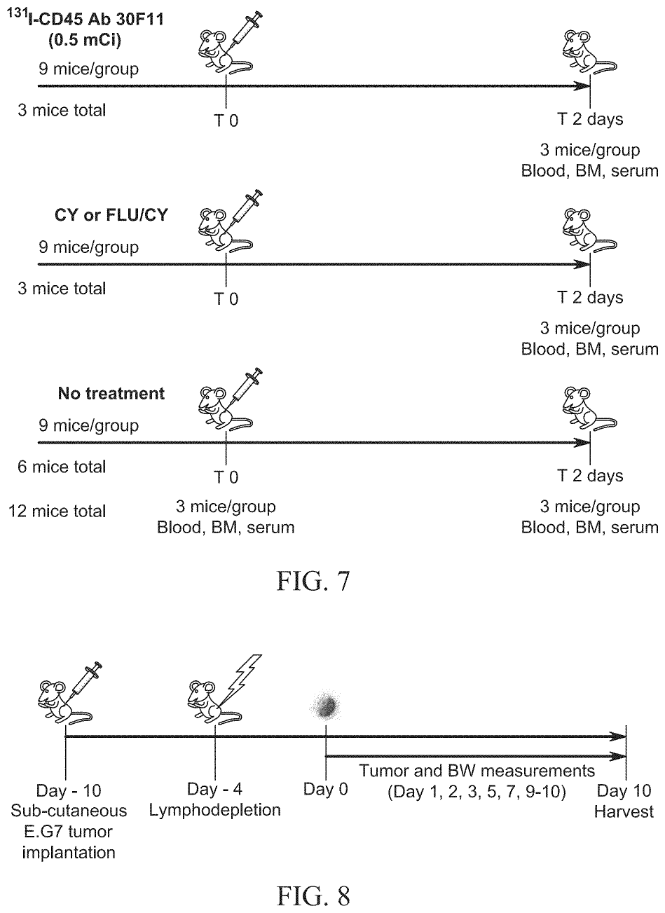

(c) At three time points (i.e., 24 hours, 48 hours and 96 hours), mice will be sacrificed and sampled (bone marrow, peripheral blood, and spleen; and serum collected). Refer to FIG. 7.

(d) Immunophenotyping will be performed on bone marrow and peripheral blood evaluating: (i) Tregs (CD4, CD25, FoxP3); (ii) CD4 & CD8 T-cells, B-cell NK cells: (CD3, CD4, CD8, CD19, CD335); (iii) HSC (Lin-, c-KIT, SCA1); and (iv) MDSC, DC, MAC, granulocytes: (CD11b, CD11c, CD244, syglec F, Ly6G, Ly6C).

(e) Cytokine profiling will be performed using Panel 31-Plex (MD31) by Luminex, including IL6, IL7, IL10, IL15, MIP1a, VEGF and IFNg.

### Example 9—Preclinical Model of Adoptive T-Cell Transfer Following Anti-CD45 Radioimmunotherapy-Mediated Conditioning/Lymphodepletion in Mice

Summary: Lymphodepletion is considered a critical step to condition a patient for receiving an autologous or allogeneic cell therapy such as CAR T. A study has been proposed to evaluate the impact of ¹³¹I-anti-CD45 radioimmunotherapy on selective depletion of immune cells and on modulation of the cytokine response in a mouse model in preparation for adoptive cell transfer. In the present study, the use of CD45 radioimmunotherapy will be evaluated as a non-myeloablative conditioning regimen prior to adoptive T-cell transfer. E.G7 lymphoma tumor-bearing mice will be conditioned by a single selected dose of ¹³¹I-anti-CD45 radioimmunotherapy prior to adoptive cell transfer of OVA-specific CD8+ T-cells and monitored for engraftment of the transferred cells and resulting anti-tumor response. The comparator will be conditioning with the chemotherapy combination of Cy with Flu or no prior conditioning. Refer to FIG. 8.

Materials: (i) Mice (5 per each group), female adolescent C57Bl/6 CD45.1 mice (3 groups; 15 mice total). (ii) Donor CD45.2 OT-1 mice (about 5 mice—sufficient for donor T-cell pool). (iii) E.G7 tumor cell line. (iv) Mouse surrogate anti-CD45 antibody 30F11 (vendor: Millipore Sigma: MABF321 200 ug, rat IgG2bκ). (v) ¹¹¹In for labeling. (vi) Fludarabine+Cyclophosphamide (treatment: 250 mg/kg Cy+50 mg/kg Flu).

Methods: (1) CD45.1 C57BL/6 mice will each be injected subcutaneously with 2×10⁶ E.G7 tumor cells until an approximate 100 mm³ tumor volume is reached (No Matrigel).

(2) Approximately 7 days post-tumor cell injection, mice will receive lymphodepletion either by ¹³¹I-anti-CD45 radioimmunotherapy (single dose I.P. of 0.5 mCi ¹³¹I-anti-CD45 antibody; 100 ug antibody in 200 ul) or Flu/Cy, or no conditioning.

(3) CD8+ T-cells will be isolated from CD45.2 OT-1 transgenic mice and cultured and activated in vitro.

(4) Four days post-lymphodepletion, 2×10⁶ CD8+ T-cells will be administered to each cohort via tail vein injection.

(5) Tumor volume and body weight will be measured daily as well as behavior and well-being assessments.

(6) Based on Hsu, et al., 2015, Oncotarget, 6:44134-44150, tumor responses should be noted within 10 days, post-T-cell administration. Following measurement on day 10, mice will be sacrificed and blood and tumors harvested.

(7) Tumors will be sectioned and stained for H&E, CD8+ cells, and Treg.

(8) Blood will be assessed by flow for the presence of engrafted CD8 cells (CD45.2+) and populations of Tregs and MDSCs.

(9) Cytokine profiling will be performed to assess terminal levels of IL-10, IL-12, IL-15 and IFNg.

### Example 10—Gene-Edited T-Cells

This example relates to lymphodepletion in cancer patients preceding administration of one or more doses of an adoptive cell therapy containing gene-edited T-cells.

CAR T-cells have shown considerable promise clinically, with response rates in refractory lymphoma patient populations exceeding 80%, and durable responses lasting six months or more in nearly 50% of treated patients.

However, these engineered T-cells remain susceptible to immune regulatory control, such as the up-regulation of immune checkpoint receptors like PD1, Lag3 or TIM3. These receptors mediate a state of exhaustion and limit the activation potential of the engineered cells. In the case of allogeneic CAR T-cells, these exogenously administered cells carry the risk of eliciting graft versus host disease (GVHD). This risk is caused by the recognition of mis-matched major histocompatibility antigens by native T-cell receptors present on the engineered allogeneic T-cells.

These adverse outcomes can be effectively mitigated through gene editing technology such as CRISPR/cas9. For example, ablation of the gene encoding PD1 would eliminate the potential for checkpoint regulation of the CAR T anti-tumor response. Further, gene editing to ablate the endogenous TCR locus in allogeneic CAR T-cell preparations would effectively prevent GVHD (Ren, et al.). Lymphodepletion is an important step in enabling successful engraftment, proliferation and persistence of administered CAR T-cells. However, safer and more effective methods for lymphodepletion (such as the subject methods) are needed to replace the use of non-specific chemotherapy and radiation. Known, non-specific regimens may contribute to the emergence of CAR T-related toxicities such as cytokine release syndrome (CRS), and gene-edited CAR T-cells are not exempt from this risk. The subject CD45-based lymphodepletion method is a safer, targeted and more effective method for depleting lymphocytes prior to gene-edited CAR T, whether the CAR T-cells are ablated for checkpoint receptors or endogenous TCRs.

### Example 11—Dosimetry of Red Marrow and Time to Reduced Activity Levels for Multicenter Pivotal Phase 3 Study of Iomab-B

Calculations were performed to evaluate times post-infusion for ¹³¹I-anti-CD45 antibody (Iomab-B) activity levels to fall to reduced or postulated “safe” levels needed to minimize radiation dose effects in red marrow to enable and facilitate follow-on cellular marrow recovery therapy.

After administration of Iomab-B, the activity of the radiolabeled antibody decreases exponentially from each of the major organs of pronounced uptake. Without wishing to be bound by theory, one possible explanation is that the dose rate to red marrow decreases exponentially over time due to the combined effects of biological clearance plus radioactive decay, such that a time point may be reached after which the total remaining dose to marrow through infinite time does not exceed a value of 25 cGy. The value 25 cGy represents one estimate of a relatively “safe” absorbed dose that should not adversely affect red marrow cellular regeneration and recovery after prior therapy with high-dose Iomab-B.

Data Review: In a subset of 25 patients who have received ¹³¹I-anti-CD45 antibody (¹³¹I-BC8) in a clinical trial, the mean initial uptake of ¹³¹I-BC8 in red marrow was 17.4 percent, which cleared with an average effective half-time of 45.1 hours. FIG. 10 shows average dose rate (cGy/hour) to red marrow in Iomab-B patients for a 100 mCi infusion. Dose rate to marrow is plotted against time-post infusion. The disappearance curve represents a single exponential function having a retention half-time of 45.1 hours. At 154 hours (about 6.5 days) post-infusion, the area-under-curve shows that during all remaining time, a total absorbed dose not to exceed 25 cGy will be imparted to red marrow (representing about 9 percent of the total dose of 271 cGy).

FIG. 10 also shows that the average initial dose rate to red marrow was 4.16 cGy/hour. Times to reach the 25 cGy point will increase with increasing activity infused. Table 4 shows the 25 cGy time points for infusion of 50 mCi, 75 mCi, 100 mCi, 150 mCi, and 200 mCi ¹³¹I-anti-CD45 antibody.

TABLE 4
Time points after which 25 cGy is delivered to red marrow,
based on observed average biokinetic parameters for 25 patients.

| | | Days | |
|---|---|---|---|
| | Time to remaining | (approximate) | Total marrow |
| ¹³¹I Activity | absorbed dose less | post-infusion | absorbed |
| Administered | than 25 cGy | (nearest half-day) | dose (cGy) |
| 50 mCi | 110 hours | 4.5 | 135 |
| 75 mCi | 136 hours | 5.5 | 203 |
| 100 mCi | 154 hours | 6.5 | 271 |
| 150 mCi | 180 hours | 7.5 | 406 |
| 200 mCi | 198 hours | 8 | 542 |

Preliminary Conclusions: From these data, it appears that a waiting period of 6 to 8 days is likely sufficient, depending on the dose administered, after infusion of the ¹³¹I-anti-CD45 antibody for start of cell-recovery therapy (ACT). For added safety, and to account for patients having bio-kinetic uptakes and clearance half-times greater than the average values, the safety time period could be increased by one or two days (to 9 to 10 days for 200 mCi) after ¹³¹I-anti-CD45 antibody infusion.

Individual patient variability: Patients differed in Iomab bio-distribution and clearance kinetics. Although the average initial uptake in red marrow was 17.4 percent of the total infusion, the highest observed red marrow uptake post-infusion was 36 percent in one patient. Whereas the average clearance half-time from marrow was 45.1 hours, the longest observed clearance half-time was 71 hours. Whereas the average absorbed dose to red marrow was 2.71 cGy per mCi ¹³¹I, the highest value observed in the current protocol was 4.24 cGy/mCi.

### Example 12—Immunophenotyping of T-Reg Cells Following ¹³¹I-BC8 Targeted Immunodepletion in Mice

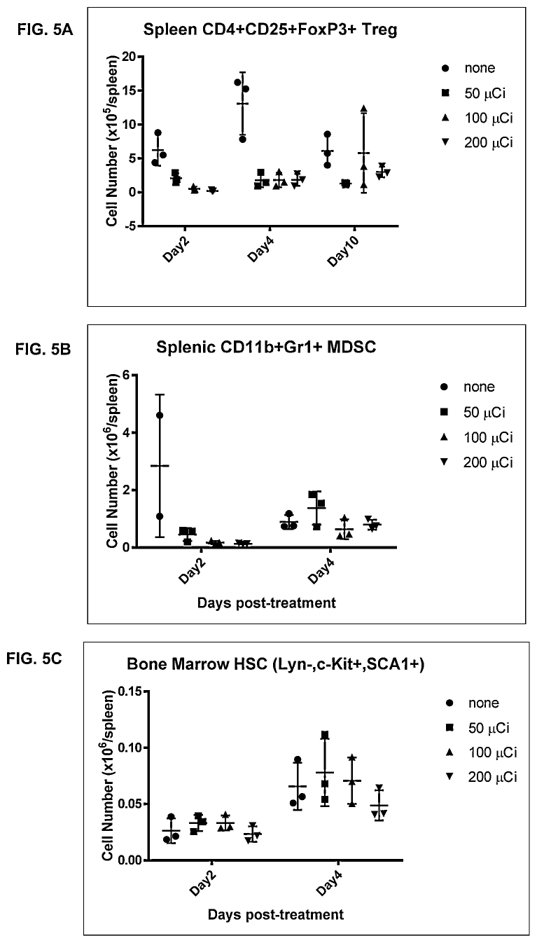

Studies of targeted immunodepletion in mice using ¹³¹I-anti-CD45 antibody directed radioimmunotherapy have shown the ability to define a dose at which lymphocytes including T-regs can be targeted for depletion, while effectively sparing an impact on the bone marrow. FIGS. 4A-4E and 5A-5C demonstrate that doses of 50-200 μCi of ¹³¹I-BC8 deplete lymphocytes and T-regs, and do not have an appreciable effect on bone marrow cells. Dose levels of 1.5-4 times higher than evaluated for lymphodepletion have been shown in a mouse tumor model of B-cell lymphoma to direct an anti-tumor effect. The lower lymphodepleting doses of this invention will also target CD45 positive tumor cells and contribute to an anti-tumor effect, reducing tumor burden including PD1 positive tumors, and improving ACT or CAR-T outcomes. In summary, when used in preparation for ACT, the invention will target the immune suppressive tumor microenvironment, leading to an improvement in ACT engraftment, response and anti-tumor outcome.

### Example 13—CD45-Targeted Immunodepletion is Safe for Adoptive Cell Therapy

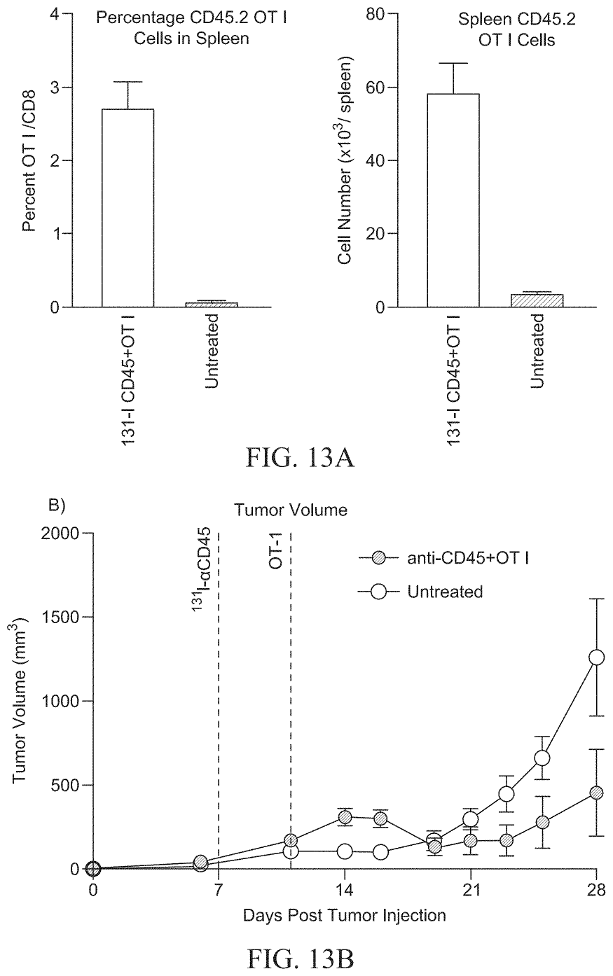

Studies of targeted immunodepletion in mice using ¹³¹I-anti-CD45 antibody directed radioimmunotherapy have shown that such forms of immunodepletion are safe prior to adoptive cell therapy. Mice having E.G7-OVA tumors engraftment were conditioned with 100 uCi of ¹³¹I-CD45 and received 1×10⁶ OT I OVA reactive T cells on day 4. As shown in FIGS. 13A and 13B, the adoptively transferred cells were able to persist in the spleen and were able to control tumor growth.

The following aspects are disclosed in this application:

Aspect 1. A method for targeted immunodepletion of specific subsets of a subject's immune cells, the method comprising: administering to the subject an effective amount of a radiolabeled antibody against CD2, CD3, CD5, CD11a, CD11b, CD11c, CD14, CD18, CD19, CD20, CD28, CD33, CD38, CD45RA, CD49a, CD52, CD96, CD116, CD122, CD132, CD152, CD154, CD244, CD272, CD305, CLA, IL-21, B7-HA, or a combination thereof; or CD19, CD20, CD33, CD38, CD45RA, CD52, or a combination thereof; or CD2, CD5, CD11a, CD18, CD49a, CD122, CD132, CD244, CD272, CD305, CLA, IL-21 receptor, and B7-HA, or a combination thereof.

Aspect 2. The method according to aspect 1, wherein the radiolabeled antibody is a bi-specific antibody comprising a lymphoid derived target and a myeloid derived target, wherein the lymphoid target is CD3, CD2, CD28, CD96, CD122, CD152, or CD154; and/or wherein the myeloid target is CD11b, CD11c, CD14, CD33, or CD116.

Aspect 3. The method according to aspect 1 or aspect 2, wherein the effective amount of the radiolabeled antibody depletes at least 50% of the targeted immune cells, or at least 70% of the targeted immune cells, or at least 80% of the targeted immune cells, and wherein the effective amount of the radiolabeled antibody depletes less than 20% of the subject's stem cells, or less than 10% of the subject's stem cells.

Aspect 4. The method according to any one of aspects 1 to 3, wherein the radiolabeled antibody is labeled with ¹³¹I, ¹²⁵I, ¹²³I, ⁹⁰Y, ¹⁷⁷Lu, ¹⁸⁶Re, ¹⁸⁸Re, ⁸⁹Sr, ¹⁵³Sm, ³²P, ²²⁵Ac, ²¹³Bi, ²¹³Po, ²¹¹At, ²¹²Bi, ²¹³Bi, ²²³Ra, ²²⁷Th, ¹⁴⁹Tb, ¹³⁷Cs, ²¹²Pb, or ¹⁰³Pd.

Aspect 5. The method according to any one of aspects 1 to 4, wherein the specific subsets of the subject's immune cells comprise cells that are lymphoid derived but are not stem cells.

Aspect 6. The method according to any one of aspects 1 to 5, wherein the specific subsets of the subject's immune cells comprise cells that are myeloid derived but are not stem cells.

Aspect 7. The method according to any one of aspects 1 to 6, wherein the radiolabeled antibody is labeled with ¹³¹I, and the effective amount is from 10 mCi to 200 mCi, or the effective amount is less than 200 mCi, or the effective amount is less than 100 mCi.

Aspect 8. The method according to any one of aspects 1 to 6, wherein the radiolabeled antibody is labeled with ²²⁵AC, and the effective amount is 0.1 μCi/kg of subject weight to 5.0 μCi/kg of subject weight.

Aspect 9. The method according to any one of aspects 1 to 8, wherein the effective amount of the radiolabeled antibody does not induce myeloablation in the subject.

Aspect 10. The method according to any one of aspects 1 to 9, wherein the effective amount of the radiolabeled antibody is administered as a single dose.

Aspect 11. The method according to any one of aspects 1 to 10, wherein the effective amount of the radiolabeled antibody provides a radiation dose of 2 Gy or less to the bone marrow.

Aspect 12. The method according to any one of aspects 1 to 11, wherein the subject is afflicted with cancer and is about to undergo adoptive cell therapy to treat the cancer.

Aspect 13. The method according to any one of aspects 1 to 12, further comprising performing adoptive cell therapy on the subject, wherein the adoptive cell therapy comprises administering to the subject an effective amount of a population of cells expressing a chimeric antigen receptor or a T-cell receptor (CAR/TCR).

Aspect 14. The method according to aspects 11 or 13, wherein the adoptive cell therapy is performed 6, 7, or 8 days after administration of the radiolabeled antibody.

Aspect 15. The method according to any one of aspects 11 to 14, wherein the adoptive cell therapy is autologous cell therapy, or wherein the adoptive cell therapy is allogeneic cell therapy.

Aspect 16. The method according to any one of aspects 11 to 15, wherein the adoptive cell therapy comprises the administration of gene-edited CAR T-cells, and wherein the gene-edited CAR T-cells fail to properly express at least one checkpoint receptor and/or at least one T-cell receptor.

Aspect 17. The method according to any one of aspects 11 to 16, wherein the population of cells expressing the CAR/TCR target CD19, CD20, CD22, CD30, CD33, CD38, CD123, CD138, CS-1, B-cell maturation antigen (BCMA), MAGEA3, MAGEA3/A6, KRAS, CLL1, MUC-1, HER2, EpCam, GD2, GPC3, GPA7, PSCA, EGFR, EGFRvIII, ROR1, mesothelin, CD33/IL3Ra, c-Met, CD37, PSMA, Glycolipid F77, GD-2, gp100, NY-ESO-1 TCR, FRalpha, CD24, CD44, CD133, CD166, CA-125, HE4, Oval, estrogen receptor, progesterone receptor, uPA, PAI-1, MICA, MICB, ULBP1, ULBP2, ULBP3, ULBP4, ULBP5 or ULBP6, or a combination thereof.

Aspect 18. The method according to any one of aspects 11 to 17, wherein the population of cells expressing the CAR/TCR target CD19, CD20, CD22, or a combination thereof.

Aspect 19. The method according to any one of aspects 1 to 11, wherein the subject is afflicted with an autoimmune disease and the specific subset of the subject's immune cells comprise B-cells, plasma cells, or a combination of both.

Aspect 20. An article of manufacture comprising (a) a radiolabeled antibody against CD2, CD3, CD5, CD11a, CD11b, CD11c, CD14, CD18, CD19, CD20, CD28, CD33, CD38, CD45RA, CD49a, CD52, CD96, CD116, CD122, CD132, CD152, CD154, CD244, CD272, CD305, CLA, IL-21, B7-HA, or a combination thereof; and (b) a label instructing the user to administer to a subject an amount of the antibody effective to deplete at least 50% of the subject's targeted immune cells and less than 20% of the subject's stem cells, or wherein the amount of the antibody is effective to deplete at least 90% of the subject's targeted immune cells and less than 10% of the subject's stem cells.

Aspect 21. The article of manufacture according to aspect 20, wherein the radiolabeled antibody is against CD19, CD20, CD33, CD38, CD45RA, CD52, or a combination thereof.

Aspect 22. The article of manufacture according to aspect 20, wherein the radiolabeled antibody is against CD2, CD5, CD11a, CD18, CD49a, CD122, CD132, CD244, CD272, CD305, CLA, IL-21 receptor, and B7-HA, or a combination thereof.

Aspect 23. The article of manufacture according to aspect 20, wherein the radiolabeled antibody is a bi-specific antibody comprising a lymphoid derived target and a myeloid derived target.

Aspect 24. The article of manufacture according to aspect 23, wherein at least one of the targets comprises CD19, CD20, CD33, CD38, CD45RA, or CD52.

Aspect 25. The article of manufacture according to aspect 23, wherein the lymphoid target is CD3, CD2, CD28, CD96, CD122, CD152, or CD154; and/or wherein the myeloid target is CD11b, CD11c, CD14, CD33, or CD116.

Aspect 26. The article of manufacture according to any one of aspects 20 to 25, wherein the antibody is radiolabeled with ¹³¹I and is administered in the amount of from 10 mCi to 200 mCi; or wherein the antibody is radiolabeled with ²²⁵Ac and is administered in the amount of from 0.1 μCi/kg to 5.0 μCi/kg of subject weight.

1. A method for targeted immunodepletion of specific subsets of a subject's immune cells, the method comprising:
administering to the subject an effective amount of a radiolabeled antibody against CD2, CD3, CD5, CD11a, CD11b, CD11c, CD14, CD18, CD19, CD20, CD28, CD33, CD38, CD45RA, CD49a, CD52, CD96, CD116, CD122, CD132, CD152, CD154, CD244, CD272, CD305, CLA, IL-21, B7-HA, or a combination thereof,
wherein the effective amount of the radiolabeled antibody depletes at least 50% of the targeted immune cells, and wherein the effective amount of the radiolabeled antibody depletes less than 20% of the subject's stem cells, and
wherein the radiolabeled antibody is labeled with ¹³¹I, ¹²⁵I, ¹²³I, ⁹⁰Y, ¹⁷⁷Lu, ¹⁸⁶Re, ¹⁸⁸Re, ⁸⁹Sr, ¹⁵³Sm, ³²P, ²²⁵Ac, ²¹³Bi, ²¹³Po, ²¹¹At, ²¹²Bi, ²¹³Bi, ²²³Ra, ²²⁷Th, ¹⁴⁹Tb, ¹³⁷Cs, ²¹²Pb, or ¹⁰³Pd.

2. The method of claim 1, wherein the radiolabeled antibody is against CD19, CD20, CD33, CD38, CD45RA, CD52, or a combination thereof.

3. The method of claim 1, wherein the radiolabeled antibody is against CD2, CD5, CD11a, CD18, CD49a, CD122, CD132, CD244, CD272, CD305, CLA, IL-21 receptor, and B7-HA, or a combination thereof.

4. The method of claim 1, wherein the specific subsets of the subject's immune cells comprise cells that are lymphoid derived but are not stem cells, or cells that are myeloid derived but are not stem cells.

5. The method of claim 1, wherein the radiolabeled antibody is a bi-specific antibody comprising a lymphoid derived target and a myeloid derived target.

6. The method of claim 5, wherein at least one of the targets comprises CD19, CD20, CD33, CD38, CD45RA, or CD52.

7. The method of claim 5, wherein the lymphoid target is CD3, CD2, CD28, CD96, CD122, CD152, or CD154, or wherein the myeloid target is CD11b, CD11c, CD14, CD33, or CD116.

8. The method of claim 1, wherein the radiolabeled antibody is labeled with ¹³¹I, and the effective amount is from 10 mCi to 200 mCi.

9. The method of claim 1, wherein the radiolabeled antibody is labeled with ²²⁵Ac, and the effective amount is 0.1 μCi/kg of subject weight to 5.0 μCi/kg of subject weight.

10. The method of claim 1, wherein the effective amount of the radiolabeled antibody does not induce myeloablation in the subject.

11. The method of claim 1, wherein the effective amount of the radiolabeled antibody is administered as a single dose.

12. The method of claim 1, wherein the effective amount of the radiolabeled antibody provides a radiation dose of 2 Gy or less to the bone marrow.

13. The method of claim 1, wherein the subject is afflicted with an autoimmune disease and the specific subset of the subject's immune cells comprise B-cells, plasma cells, or a combination of both.

14. The method of claim 1, wherein the subject is afflicted with cancer and is about to undergo adoptive cell therapy to treat the cancer.

15. The method of claim 1, further comprising:
performing adoptive cell therapy on the subject, wherein the adoptive cell therapy comprises administering to the subject an effective amount of a population of cells expressing a chimeric antigen receptor or a T-cell receptor (CAR/TCR).

16. The method of claim 15, wherein the adoptive cell therapy is performed 6, 7, or 8 days after administration of the radiolabeled antibody.

17. The method of claim 15, wherein the adoptive cell therapy is autologous cell therapy, or allogeneic cell therapy.

18. The method of claim 15, wherein the adoptive cell therapy comprises the administration of gene-edited CAR T-cells, and wherein the gene-edited CAR T-cells fail to properly express at least one checkpoint receptor and/or at least one T-cell receptor.

19. The method of claim 15, wherein the population of cells expressing the CAR/TCR target CD19, CD20, CD22, CD30, CD33, CD38, CD123, CD138, CS-1, B-cell maturation antigen (BCMA), MAGEA3, MAGEA3/A6, KRAS, CLL1, MUC-1, HER2, EpCam, GD2, GPC3, GPA7, PSCA, EGFR, EGFRvIII, ROR1, mesothelin, CD33/IL3Ra, c-Met, CD37, PSMA, Glycolipid F77, GD-2, gp100, NY-ESO-1 TCR, FRalpha, CD24, CD44, CD133, CD166, CA-125, HE4, Oval, estrogen receptor, progesterone receptor, uPA, PAI-1, MICA, MICB, ULBP1, ULBP2, ULBP3, ULBP4, ULBP5 or ULBP6, or a combination thereof.

20. The method of claim 15, wherein the population of cells expressing the CAR/TCR target CD19, CD20, CD22, or a combination thereof.

21-26. (canceled)
